(19) United States
(12) Patent Application Publication
(10) Pub. No.: US 2024/0066037 A1
(43) Pub. Date: Feb. 29, 2024

(54) CATHEPSIN INHIBITORS FOR PREVENTING OR TREATING VIRAL INFECTIONS

(71) Applicants: GLAXOSMITHKLINE INTELLECTUAL PROPERTY DEVELOPMENT LIMITED, Stevenage (GB); THE GOVERNMENT OF THE UNITED STATES AS REPRESENTED BY THE SECRETARY OF THE ARMY, Frederick, MD (US); THE HENRY M. JACKSON FOUNDATION FOR THE ADVANCEMENT OF MILITARY MEDICINE, INC., Bethesda, MD (US)

(72) Inventors: Sina BAVARI, Collegeville, PA (US); Christopher D. KANE, Collegeville, PA (US); Andrew James PEAT, Collegeville, PA (US); Veronica SOLOVEVA, Collegeville, PA (US)

(21) Appl. No.: 17/913,870

(22) PCT Filed: Mar. 26, 2021
(86) PCT No.: PCT/IB2021/052548

§ 371 Date: Sep. 23, 2022

Related U.S. Application Data
(60) Provisional application No. 63/000,487, filed on Mar. 26, 2020.

(51) Int. Cl. A61K 31/55; A61K 45/06; A61P 31/14

(52) U.S. Cl. CPC A61K 31/55; A61K 45/06; A61P 31/14

(57) ABSTRACT

Provided are cathepsin inhibitor compounds and pharmaceutically acceptable salts thereof, and combinations thereof, for use in the treatment or prophylaxis of viral disease in an animal.

### NAMES OF PARTIES TO A JOINT RESEARCH AGREEMENT

The invention described herein is the subject of a Collaboration Research and Development Agreement (CRADA) between GlaxoSmithKline LLC (GSK) and the United States Army Medical Research Institute of Infectious Diseases (USAMRIID) entered into on Oct. 22, 2015, and a Collaboration Research and Development Agreement (CRADA) between GSK and USAMRIID entered into on Dec. 21, 2018.

This invention was made with government support under W81XWH-16-0019 and W81XWH-19-0083 awarded by the United States Army Medical Research Institute of Infectious Diseases, a subordinate command of the United States Army Medical Research and Development Command. The government has certain rights in the invention.

### SEQUENCE LISTING

The instant application contains a Sequence Listing, which has been submitted electronically in computer readable form in .txt format and is hereby incorporated by reference in its entirety. Said .txt file, created on Sep. 6, 2023, is named PU66872US SEQ LIST ST25 Corrected 5Sept2023.bd and is 1,653 bytes.

### FIELD OF THE INVENTION

The present invention relates to certain cathepsin inhibitor compounds, methods and pharmaceutical compositions for preventing and/or treating viral disease cause by a virus. Methods for preparing such compounds and methods of using the compounds are also disclosed. In particular, the treatment and prophylaxis of viral disease caused by viruses in the family Coronaviridae and Filoviridae, among others, are disclosed.

### BACKGROUND OF THE INVENTION

Every year viruses bring about the deaths of millions of people around the world. (See, e.g., Fact Sheets on HIV/AIDS, Hepatitis C Virus, and Influenza, WORLD HEALTH ORGANIZATION). The diseases caused by viral infection are numerous and diverse, as are the structures, sizes, genomes, and infection cycles of the viruses that cause them; facts which tend to complicate and fracture research efforts in this area. Viruses are classified by evaluating several characteristics, including the type of viral genome. Viral genomes can be comprised of DNA or RNA, can be double-stranded or single-stranded (which can further be positive-sense, negative-sense, or ambi-sense), and can vary greatly by size and genomic organization.

By way of one example, Ebola hemorrhagic fever is a disease caused by one of five different Ebola viruses. Four of the strains can cause severe illness in humans and animals. The fifth, Reston virus, has caused illness in some animals, but not in humans. Ebola viral disease, which has a case fatality rate of up to 90%, is a severe acute viral illness often characterized by the sudden onset of fever, intense weakness, muscle pain, headache, nausea and sore throat. This is followed by vomiting, diarrhea, impaired kidney and liver function, and in some cases, both internal and external bleeding. Laboratory findings frequently include low white blood cell and platelet counts and elevated liver enzymes. Such symptoms typically occur between two and twenty-one days after the initial infection.

In 2014, the largest outbreak in history of Ebola occurred in West Africa and resulted in over 28,000 cases before it subsided a year later. See, Warren, et al., Nature Vol. 531, p. 381-389 (2016). Two out of five people who acquired Ebola during this outbreak died. Moreover, there was a marked absence of market approved medications that were specifically targeted to the Ebola virus. Instead, the vast majority of treatments were confined to simple palliative care. See, Centers for Disease Control and Prevention, website, Ebola Outbreaks, 2014. https://www.cdc.gov/vhf/ebola/outbreaks/2014-west-africa/

In 2018, there was another outbreak of Ebola, this time in the Eastern Democratic Republic of Congo (DRC), which spread to Uganda and is currently ongoing. This second major Ebola outbreak has resulted in 2592 confirmed cases and 1743 deaths as of July 2019.

From the end of 2014 into 2016, a formerly dormant and/or mild mosquito-borne virus known as the Zika virus exploded in Brazil, spreading within a year to nearly every country in Latin America and the Caribbean that was home to the mosquito species Aedes aegypti. Zika virus infections typically produce a mild or symptomless disease in infected persons, but some cases have led to an increase in reports of Guillain-Barré syndrome after infection with Zika virus—a rare neurological disorder—with lasting impact. More troublesome, in 2015 Brazil reported to the WHO that Zika was linked to insidious and troublesome adverse consequences in certain cases, particularly in infants born to mothers who were infected with the Zika virus during pregnancy. The adverse consequences seen were microencephaly and severe brain abnormalities. By early 2016, the WHO reported that the association between Zika and microencephaly cases and other neurological disorders was a Public Health Emergency of International Concern and concluded that scientific consensus supported Zika virus as the cause of the microencephaly and Guillain-Barré syndrome disorders being reported in countries with Zika outbreaks (see https://www.who.int/emergencies/zika-virus/articles/one-year-outbreak/en/index1.html for further discussion). As of July 2018, Zika virus infection has been reported in 86 countries, with no known treatment for the virus or its associated neurological diseases (https://www.who.int/news-room/fact-sheets/detail/zika-virus).

The emergence of highly contagious, highly morbid coronavirus infections has also resulted in multiple global health crises in the past 20-30 years. In 2003, a multi-country outbreak of atypical pneumonia of unknown etiology emerged and was referred to as Severe Acute Respiratory Syndrome (SARS). SARS was linked to a new strain of coronavirus (https://www.cdc.gov/mmwr/preview/mmwrhtml/mm5214a1.htm) and ultimately named SARS-CoV. SARS-CoV resulted in 8,098 persons infected and 774 deaths reported (https://www.cdc.gov/mmwr/preview/mmwrhtml/mm5249a2.htm) in 29 countries.

In September 2012, a patient with a severe respiratory illness was reported in Saudi Arabia. The disease was identified as a sever complication of a new coronavirus. Tracking back, it was determined that the first case of a disease that would later be named Middle East Respiratory Syndrome (MERS) occurred in April, 2012 in Jordan, and the virus responsible for MERS was named MERS-CoV. MERS results in severe respiratory illness, with patients exhibiting fever, cough and severe shortness of breath. The morbidity of MERS has been determined to be nearly 35%, with 3-4 of every 10 cases of MERS resulting in death. By late 2019, 2494 confirmed cases of MERS reported, with 858 deaths, (https://www.who.int/emergencies/mers-cov/en/), with most of these cases and deaths reported in Saudi Arabia. To date, all reported cases MERS have been linked to travel to, from, through or residence in or near the Arabian Peninsula, although 27 countries have reported cases of MERS-CoV.

Recently, in the last week of December 2019, another new coronavirus-related disease was reported in Wuhan, China. On Jan. 30, 2020 WHO declared COVID-19 a Public Health Emergency of International Concern (https://www.who.int/emergencies/diseases/novel-coronavirus-2019/events-as-they-happen). The coronavirus behind this new viral disease originally referred to as 2019-nCoV, and the disease was named COVID-19 on Feb. 11, 2020 by the World Health Organization (https://www.who.int/emergencies/diseases/novel-coronavirus-2019/events-as-they-happen). Also on Feb. 11, 2020, the virus responsible for COVID-19 was officially re-named SARS-CoV-2 by the International Committee Taxonomy of Viruses (ICTV) (https://talk.ictvonline.org/). On Mar. 11, 2020, WHO declared COVID-19 a pandemic (https://www.who.int/dg/speeches/detail/who-director-general-s-opening-remarks-at-the-media-briefing-on-covid-19---11-march-2020).

Since December 2019, COVID-19 has spread from China to 190 countries, with over 531,000 confirmed cases and over 24,000 deaths reported (https://experience.arcgis.com/experience/685d0ace521648f8a5beeeee1b9125cd) (numbers current as of 11 pm edt, Mar. 26, 2020), far outpacing in scope and severity the impact seen with the SARS-CoV coronavirus outbreak of 2003 (https://www.ibtimes.sg/drawing-parallels-sars-mers-wuhan-coronavirus-38517), although COVID-19, with a mortality rate of approximately 2% (https://www.who.int/csr/sars/archive/2003_05_07a/en/), is less deadly than SARS or MERS (https://www.hindustantimes.com/world-news/who-warns-against-novel-coronavirus-blanket-measures/story-bbTICTA9xwzC8fSWK4d9CN.html_). SARS has a mortality rate of ˜15% (https://www.who.int/csr/sars/archive/2003_05_07a/en/), and MERS has a mortality rate of ˜35% (https://www.who.int/news-room/q-a-detail/middle-east-respiratory-syndrome-coronavirus-(mers-cov)).

Coronaviruses consist of an enveloped single strand positive sense RNA genome of 26 to 32 kb in length. They are classified by phylogenetic similarity into four categories: α (e.g. 229E and NL-63), β (e.g. SARS-CoV-2, SARS-CoV, MERS-CoV, HKU1, and OC43), γ and δ. SARS-CoV-2 has also been reported to have 79% sequence identity to SARS-CoV-1, however certain regions of the SARS-CoV-2 genome exhibit greater or lesser degrees of conservation to SARS-CoV-1.

Coronaviruses, including SARS-CoV-2, utilise a membrane bound spike (S) protein to bind to a host cell surface receptor, ACE2, to gain cellular entry. The trimeric S protein contains two subunits, S1 and S2. The S1 subunit contains a fragment called the receptor-binding domain (RBD) that is able to bind ACE2. Following entry into the host cell, the RNA genome is translated into two large polypeptides by the host ribosomal machinery and several smaller polypeptides. The two large polypeptides are processed by two proteases, the coronavirus main proteinase (3C-Like) and the papain-like proteinase to generate the proteins required for viral replication and packaging.

Several clades of SARS-CoV-2 (strains) have been identified which are designated L (original strain from Wuhan), S (named after the L to S amino acid change—the ORF8:L84S mutation), G (named after the D to G amino acid change in the Spike protein—the S:D614G mutation), V (named after the G to V amino acid change—the ORF3a:G251V mutation), and O (sequences not matching any of these criteria for the other clades). Clade G comprises two derivative clades, GH (characterized by the ORF3a:Q57H mutation) and GR (having a N:RG203KR mutation). Generally, clades G and GR are prevalent in Europe, and clades S and GH have been mostly observed in the Americas. The L Glade is mostly represented by sequences from Asia. At present, clades G and its derivative offspring clades GH and GR are the most common among the sequences SAR-CoV-2 genomes, accounting for 74% of all world sequences, globally. The GR Glade, having both the Spike D614G and Nucleocapsid RG203KR mutations, is the most common representative of the SARS-CoV-2 genome population worldwide. The original viral strain, Glade L, continues to account for 7% of the sequenced genomes, and clades S and V have similar frequencies in the global dataset of sequences. For a somewhat different assessment of SARS-CoV-2 clades, see Li, T., Liu, D., Yang, Y. et al. Phylogenetic supertree reveals detailed evolution of SARS-CoV-2. Sci Rep 10, 22366 (2020). https://doi.org/10.1038/s41598-020-79484-8.

Although several groups have confirmed the relatively low variability of SARS-CoV-2 genomes, it is not clear if the different fatality rates or speed of transmission observed within different countries is related to differences in virulence between different clades. Several new variants have recently emerged in the UK (201/501Y.V1/B.1.1.7), South Africa (20H/501Y.V2/6.1.351), Brazil (P.1/203/501Y.V3/13.1.1.248) and a novel variant in California descended from cluster 20C, defined by 5 mutations (ORF1a: I4205V, ORF1b:D1183Y, S:S13I; W152C; L452R) and designated CAL.20C (20C/S;452R;B.1.429).

Diverse SARS-CoV-2 vaccine types are currently under development, including inactivated vaccines, nucleic acid vaccines, adenovirus-based vector vaccines, and recombinant subunits vaccines. The majority of vaccines comprise or encode a portion or the whole of the S protein. There is significant concern however, that these vaccines may not provide adequate protection against newly arising variants of SARS-CoV-2 including mutations in the S protein.

Alternative agents that reduce viral entry and replication of SARS-CoV-2 will have potential to reduce the magnitude of viral infection and may provide benefits in this rapid evolving pandemic.

Accordingly, there is an ongoing need in the art for medications to treat or prevent certain viral diseases, including, but not limited disease caused by coronaviruses, Ebola viruses, Dengue virus, and mosquito-borne viruses such as Zika (ZIKV) and West Nile virus, that are safe and efficacious.

Cathepsins function in the normal physiological process of protein degradation in animals, including humans, e.g., in the degradation of connective tissue. However, elevated levels of these enzymes in the body can result in pathological conditions leading to disease. In particular, one cathepsin K inhibitor has been reported to treat certain parasitic disease and/or bone or cartilage degradation. See, PCT published patent application No. WO 2001070232 and PCT published patent application No. WO2002017924 , and Wang, et al., Tetrahedron, 65:32 (2009). Cathepsin K has been variously denoted as cathepsin O or cathepsin 02 in the literature. However, the designation cathepsin K is considered to be the most appropriate one. Thus, cathepsins have been implicated as causative agents in various disease states.

Cathepsin L, also known as cathepsin L1, is encoded by the CTSL1 gene. It is a lysosomal cysteine protease responsible for protein degradation and involved in a variety of cellular processes including bone degradation and angiogenesis. It also has a role in degrading antigenic peptides during MHC class II antigen presentation. If present extracellularly, it is known to degrade the extracellular matrix including collagen and elastin proteins. It is also reported to be involved in the cleavage and maturation of viral proteins including those of Ebola virus.

Cathepsin B is a lysosomal cysteine protease encoded by the CTSBgene. Cathepsin B is important for both intracellular and extracellular proteolysis and is associated with a wide variety of illnesses including bone degradation, cancer and Alzheimer's disease.

Many viruses hijack host cellular machinery to enable infection, replication, and spread. It is known that members of the human cysteine protease family referred to as cathepsins are responsible for activation of proteins from several viruses (Zhou et al., Protease inhibitors targeting coronavirus and filovirus entry, Antiviral Research, 2015). For example, host cathepsins are able to cleave and activate the Ebola virus glycoprotein (K Schornburg et al, J Virol (2006) 80, 4174) and the SARS coronavirus spike protein (B Bosch et al, J Virol (2008) 82, 8887). More recently, it was reported that there may be a possible dual function of the catabolic route at play in host infection by the Zika virus: cooperation between autophagy and innate immunity, and the suppression of innate immunity by autophagy (Shoji-Kawat et al. Int. Immunopharmaco.l, 2014; 18:55-65). Thus, compounds that inhibit later stages of autophagy—the degradation of autophagosome content by lysosome enzymes—might be effective agents against viruses such as Zika virus (ZIKV) and Dengue virus, including compounds which inhibit the activity of lysosomal cathepsin B, possibly by binding directly to the enzyme (Gratton et al., Int J. Mol. Sci, 2019 March; 20(5):1048 and Balasubramian et al., Antiviral Res. 2017; vol. 137; pp. 141-159). Cathepsin B has been reported to be involved in the cleavage and maturation of viral proteins, including those of Ebola virus. Moreover, SARS-CoV is known to mediate receptor binding and entry into cells by its spike (S) glycoprotein and this process is inhibited by cathepsin L inhibitors (Simmons, G. et al. “Inhibitors of cathepsin L prevent severe acute respiratory syndrome coronavirus entry” PNAS Aug. 16, 2005 102 (33) 11876-11881; https://doi.org/10.1073/pnas.0505577102) There is a need to identify inhibitors that can modulate such processes and interfere with the viral replication cycle, thereby reducing the symptoms of disease.

### SUMMARY OF THE INVENTION

An object of the present invention is to provide a method for the treatment or prophylaxis of a viral disease in a subject caused by a virus, the method comprising administering to a subject in need thereof a therapeutically effective amount of an inhibitor of a cysteine protease, even more particularly administering a therapeutically effective amount of an inhibitor of the papain cysteine protease superfamily, still more particularly administering a therapeutically effective amount of an inhibitor of a cysteine protease of the cathepsin family. Relacatib (SB-462795) is a potent and orally bioavailable small molecule inhibitor of cathepsin K that inhibits bone resorption both in vitro in human tissue and in vivo in cynomolgus monkeys. SB-462795 is a potent inhibitor of human cathepsins K, L, and V (Ki, app=41, 68, and 53 pM, respectively) that exhibits 39-300-fold selectivity over other cathepsins (see S. Kumar et al., Bone (2007), vol 40 (1), pp.122-131) Epub 2006 Sep. 7. Relacatib (SB-462795) is also a potent inhibitor of cathepsin B (Cat B) with a Kᵢ of 15 nM, 11 nM and 6.7 nM for human, monkey and rate Cat B, respectively (see Yamashita, et al. , J. Med. Chem. (2006), 49, 1597-1612, and Table 1 below). Therefore, it is an object of the invention to provide a method for the treatment or prophylaxis of a viral disease in a subject caused by a virus, the method comprising administering to a subject in need thereof a therapeutically effective amount of SB-462795 (relacatib), or a pharmaceutically acceptable salt thereof.

One aspect of the invention provides a method for the treatment or prophylaxis of a viral disease in a subject in need thereof caused by a virus, the method comprising administering to the subject a therapeutically effective amount of a compound of the structure:

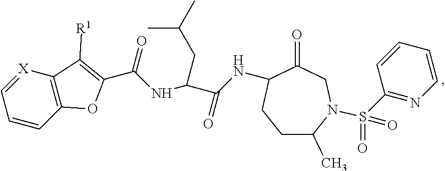

or a pharmaceutically acceptable salt thereof,
wherein:
X is selected from N or CR²; and
R¹ and R² is each independently selected from —H or —(C₁-C₈)alkyl.

One aspect of the invention provides a method for the treatment or prophylaxis of a viral disease in a subject in need thereof caused by a virus, the method comprising administering to the subject a therapeutically effective amount of a compound of the structure:

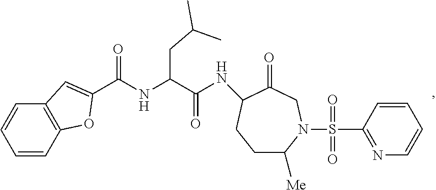 [SMILES: CC(C)CC(NC(=O)c1cc2ccccc2o1)C(=O)NC1CCC(C)N(S(=O)(=O)c2ccccn2)CC1=O]

or a pharmaceutically acceptable salt thereof.

A particular aspect of the invention provides a method for the treatment or prophylaxis of a viral disease in a subject in need thereof caused by a virus, the method comprising administering to the subject a therapeutically effective amount of a compound of the structure, wherein the compound is:

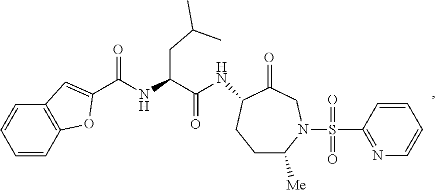 [SMILES: CC(C)C[C@H](NC(=O)c1cc2ccccc2o1)C(=O)N[C@H]1CC[C@@H](C)N(S(=O)(=O)c2ccccn2)CC1=O]

or a pharmaceutically acceptable salt thereof.

One aspect of the invention provides a compound of the structure:

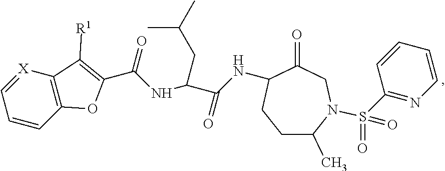

or a pharmaceutically acceptable salt thereof,
wherein:
X is selected from N or CR²; and
R¹ and R² is each independently selected from —H or —(C₁-C₈)alkyl, for use in antiviral therapy.

One aspect of the invention provides a compound which is

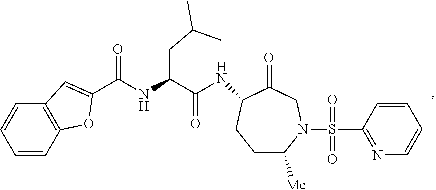 [SMILES: CC(C)C[C@H](NC(=O)c1cc2ccccc2o1)C(=O)N[C@H]1CC[C@@H](C)N(S(=O)(=O)c2ccccn2)CC1=O]

or a pharmaceutically acceptable salt thereof, for use in antiviral therapy.

One aspect of the invention provides a first antiviral compound which is

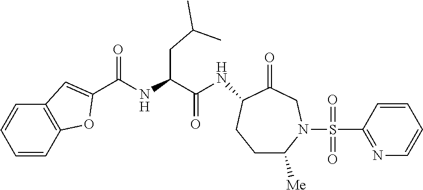 [SMILES: CC(C)C[C@H](NC(=O)c1cc2ccccc2o1)C(=O)N[C@H]1CC[C@@H](C)N(S(=O)(=O)c2ccccn2)CC1=O]

or a pharmaceutically acceptable salt thereof, in combination with a second antiviral compound, for use in antiviral therapy.

One aspect of the invention provides a compound of the structure:

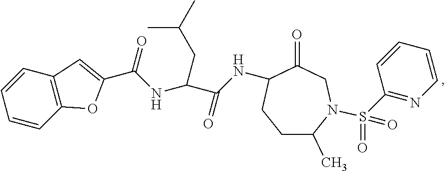 [SMILES: CC(C)CC(NC(=O)c1cc2ccccc2o1)C(=O)NC1CCC(C)N(S(=O)(=O)c2ccccn2)CC1=O]

or a pharmaceutically acceptable salt thereof, for use in the treatment or prophylaxis of a viral disease in an animal caused by a virus, wherein the virus is a poliovirus, a rhinovirus, a coxsackievirus, a foot-and-mouth virus (FMDV)), an influenza virus (influenza A virus, influenza B virus, influenza C virus, an avian influenza virus), a flavivirus, a hepatitis virus (hepatitis A virus, hepatitis B virus, hepatitis C virus, hepatitis D virus, hepatitis E virus), a coronavirus (CoV-SARS virus, coronavirus responsible for Middle Eastern Respiratory Syndrome (MERS), CoV-SARS-2), an Ebola virus, a Marburg virus, a Nipah virus (NiV), a Hendra virus, an arenavirus, a Rift Valley Fever virus, a yellow fever virus, a respiratory syncytial virus (RSV), a West Nile virus, a Dengue fever virus, an Aichi virus, an enterovirus, a rubella virus, a Theiler's murine encephalomyelitis virus (TMEV), an echovirus or a human immunodeficiency virus (HIV).

One aspect of the invention provides a compound which is
N-((2S)-4-methyl-1-(((4S,7R)-7-methyl-3-oxo-1-(pyridin-2-ylsulfonyl)azepan-4-yl)amino)-1-oxopentan-2-yl)benzofuran-2-carboxamide;
3-methyl-N-((S)-4-methyl-1-(((4S,7R)-7-methyl-3-oxo-1-(pyridin-2-ylsulfonyl)azepan-4-yl)amino)-1-oxopentan-2-yl)furo[3,2-b]pyridine-2- carboxamide,
   or a pharmaceutically acceptable salt thereof, for use in the treatment or prevention of COVID-19.

In one embodiment, there is provided use of a compound which is:
N-((2S)-4-methyl-1-(((4S,7R)-7-methyl-3-oxo-1-(pyridin-2-ylsulfonyl)azepan-4-yl)amino)-1-oxopentan-2-yl)benzofuran-2-carboxamide;
3-methyl-N-((S)-4-methyl-1-(((4S,7R)-7-methyl-3-oxo-1-(pyridin-2-ylsulfonyl)azepan-4-yl)amino)-1-oxopentan-2-yl)furo[3,2-b]pyridine-2- carboxamide,
   or a pharmaceutically acceptable salt thereof, for the treatment or prevention of COVID-19 in a subject, e.g. a human.

In one embodiment, the subject is infected with SARS-CoV-2.

The invention also provides a method of treatment of a subject with COVID-19 with a therapeutically effective amount of a compound selected from:
N-((2S)-4-methyl-1-(((4S,7R)-7-methyl-3-oxo-1-(pyridin-2-ylsulfonyl)azepan-4-yl)amino)-1-oxopentan-2-yl)benzofuran-2-carboxamide;
3-methyl-N-((S)-4-methyl-1-(((4S,7R)-7-methyl-3-oxo-1-(pyridin-2-ylsulfonyl)azepan-4-yl)amino)-1-oxopentan-2-yl)furo[3,2-b]pyridine-2- carboxamide,
   or a pharmaceutically acceptable salt thereof.

In one embodiment the subject is a human.

In addition, the invention provides a method for identifying subjects to be treated in accordance with the methods described herein, the method comprising a step of assaying a specimen from a subject for the presence of SARS-CoV-2 RNA. In some embodiments, the method is capable of identifying L strain SARS-CoV-2 RNA, S strain SARS-CoV-2 RNA, G strain SARS-CoV-2 RNA, GH strain SARS-CoV-2 RNA, GR strain SARS-CoV-2 RNA, V strain SARS-CoV-2 RNA, or O strain SARS-CoV-2 RNA. In some embodiments, where SARS-CoV-2 RNA is detected, the method may further comprises a treatment step as described herein.

In specific embodiments, the invention provides treatment for particular populations of patients with COVID-19, for example a patient or subject infected with SARS-CoV-2 i.e. a patient or subject who test positive to SARS-Co-2, a patient or subjects in a high risk categories and a patient or subject with a secondary conditions. In particular embodiments, the patient or subject has pneumonia or acute respiratory distress disorder. In additional embodiments, the patient or subject is additionally undergoing extra-corporeal membrane oxygenation, mechanical ventilation, non-invasive ventilation, receiving oxygen therapy or receiving antiviral or steroid treatment.

In one embodiment, the uses and methods described herein are for treatment of a patient infected with a strain (clade) of SARS-CoV-2 selected from the L strain (clade), the S strain (clade), the G strain (clade), the GH strain (clade), the GR strain (clade), the V strain (clade) or the O strain (clade) of SARS-CoV-2.

In one embodiment, the uses and methods described herein are for treatment of a patient infected with the L strain (clade) of SARS-CoV-2 or a variant thereof. In another embodiment, the uses and methods described herein are for treatment of a patient infected with the S strain (clade) of SARS-CoV-2, or a variant thereof. In another embodiment, the uses and methods described herein are for treatment of a patient infected with the G strain (clade) of SARS-CoV-2, or a variant thereof.

In another embodiment, the uses and methods described herein are for treatment of a patient infected with the GH strain (clade) of SARS-CoV-2, or a variant thereof. In another embodiment, the uses and methods described herein are for treatment of a patient infected with the GR strain (clade) of SARS-CoV-2, or a variant thereof. In another embodiment, the uses and methods described herein are for treatment of a patient infected with the V strain (clade) of SARS-CoV-2, or a variant thereof. In another embodiment, the uses and methods described herein are for treatment of a patient infected with the O strain (clade) of SARS-CoV-2, or a variant thereof. In a particular embodiment the uses and methods described herein are for treatment of a subject infected with a variant of SARS-CoV-2, including the UK variant (201/501Y.V1/B.1.1.7), the South Africa variant (20H/501Y.V2/B.1.351), the Brazil variant (P.1/20J/501Y.V3/13.1.1.248) and the novel California variant descended from cluster 20C, defined by 5 mutations (ORF1a:I4205V, ORF1b:D1183Y, S:S13I; W152C; L452R) and designated CAL.20C (20C/S;452R;B.1.429).

In another embodiment, the subject is not infected with SARS-CoV-2 such that the use is for prevention of COVID-19. Subjects suitable for such prophylactic use include subjects in high risk categories, health care professionals and close contacts of subjects infected with SARS-CoV-2.

One aspect of the invention provides use of a compound or a pharmaceutically acceptable salt thereof having the structure

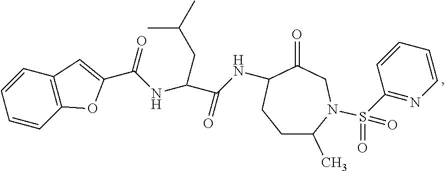 [SMILES: CC(C)CC(NC(=O)c1cc2ccccc2o1)C(=O)NC1CCC(C)N(S(=O)(=O)c2ccccn2)CC1=O]

or a pharmaceutically acceptable salt thereof, in the manufacture of a medicament for use in treatment of a viral disease caused by a virus, wherein the virus is a poliovirus, a rhinovirus, a coxsackievirus, a foot-and-mouth virus (FMDV), an influenza virus (influenza A virus, influenza B virus, influenza C virus, an avian influenza virus), a flavivirus, a hepatitis virus (hepatitis A virus, hepatitis B virus, hepatitis C virus, hepatitis D virus, hepatitis E virus), a coronavirus (SARS-CoV, SARS-CoV-2, a coronavirus responsible for Middle Eastern Respiratory Syndrome (MERS) virus), an Ebola virus, a Marburg virus, a Nipah virus (NiV), a Hendra virus, an arenavirus, a Rift Valley Fever virus, a yellow fever virus, a respiratory syncytial virus (RSV), a West Nile virus, a Dengue fever virus, an Aichi virus, an enterovirus, a rubella virus, a Theiler's murine encephalomyelitis virus (TMEV), an echovirus, or a human immunodeficiency virus (HIV).

Also provided is a method for the treatment or prophylaxis of a viral disease in a subject caused by a virus, the method comprising administering to the subject in need thereof a pharmaceutical composition comprising a therapeutically effective amount of a compound disclosed herein, particularly SB-462795, or a pharmaceutically acceptable salt thereof, together with a pharmaceutically acceptable carrier or excipient.

### DESCRIPTION OF DRAWINGS/FIGURES

FIG. 1 shows relacatib (SB-462795) and computational models of cathepsin binding:

FIG. 1A shows (A) Structural alignment of human CatL (blue, darker) and CatB (rose, lighter) yielding a Cα root-mean-square deviation of 1.82 Å (angstrom) and a sequence identity of 24.8%.

FIG. 1B shows Docked poses of SB-462795 bound to the molecular surface of human CatL (blue); the top-ranked pose is colored white. The surface region colored yellow (light lower right region) is the residue Leu 144 and is the variant with the query sequence from Macaca mulatta containing the residue Met.

FIG. 1C shows the top-ranked pose of SB-462795 bound to shape-filling model of human CatL;

FIG. 1D shows the top-ranked docked pose of SB-462795 bound to shape-filling model of human CatB;

FIG. 1E shows Top-ranked conformational poses of SB-462795 for human CatL (green, foreground left, upper middle), human CatB (rose, background left, uppermiddle) and human CatK (cyan, right upper separate ring)

FIG. 2 shows broad spectrum antiviral activity of SB-462795. Antiviral activity was assessed in cell-based high-content assays using the indicated virus and cell combinations:

FIG. 2A through 2J show virus infected cells in the absence (DMSO alone, left-hand panel) and presence of SB-462795 (center or right panel, concentration indicated).

FIGS. 2K through 2N shows imaging assays wherein green (lightest imaged regions) represents antibody directed against the indicated viral coat protein, Blue (less light imaged regions) represents Hoechst dye;

FIG. 2K shows Ebola Makona;

FIG. 2I shows Marburg virus;

FIG. 2M shows MERS coronavirus;

FIG. 2N shows CoV-SARS;

FIG. 2O Shows Nipah virus, which forms syncytia during in vitro infection of HeLa cells

FIG. 3 shows SB-462795 pharmaceutics in the absence and presence of Ebola virus (EBOV) infection. Pharmacokinetic parameters were calculated for SB-462795 in multiple ascending dose studies performed in rats (black), rhesus macaques (red), and normal healthy humans in the absence of infection (blue).

FIG. 3A shows area under the plasma concentration versus time curve (AUC) on the ordinate and dose on the abscissa.;

FIG. 3B shows plasma half-life on the ordinate versus dose on the abscissa

FIG. 3C shows plasma concentration maximum post dose on the ordinate versus dose on the abscissa. (Cₘₐₓ);

FIG. 3D shows plasma concentration minimum or trough level on the ordinate versus dose on the abscissa. (Cₘᵢₙ);

FIG. 3E shows SB-462795 plasma exposure in rhesus macaques infected with EBOV. The drug was orally administered by gavage once per day for 14 days. Dot plots indicate individual exposure levels with the group mean (vertical bars) and standard deviations shown in black.

FIG. 4 shows SB-462795 post-exposure protection against EBOV in rhesus macaques.

FIG. 4A shows clinical signs of disease in individual rhesus macaques exposed to Ebola virus. Animals were observed multiple times each day by a blinded evaluator and were subjectively assigned a clinical disease score ranging from 0-5 based on responsiveness, posture and activity. Maximum daily sores were converted to a color with darker colors corresponding to more severe disease signs. The schematic was truncated to highlight clinical scores during days 1-4.

FIG. 4B shows Kaplan-Meier survival curves. *P=0.0565 for SB-462795 treatment (blue) versus vehicle control (black) groups using Log-ran Mantel-Cox test.

FIG. 4C shows dot plots for individual plasma viral RNA concentrations in treatment (blue) vs, vehicle (black).

FIG. 4D through FIG. 4I show clinical pathology values for SB-462795 treatment (blue) and vehicle (black) for selected analytes including d platelets, e activated partial thromboplastic time (APTT), f fibrinogen, g C-reactive protein (CRP), h blood urea nitrogen (BUN), i aspartate aminotransferase (AAT). FIG. 5 shows signs of Ebola virus present in control and SB-462795-treated monkey (rhesus macaque) survivors:

FIG. 5A shows hepatocyte necrosis in liver;

FIG. 5B shows fibrosis, necrosis and depletion of lymphoid follicles in spleen of (rhesus macaques);

FIG. 5C shows enhanced GI pathology noted in colon of both control and SB-462795-treated animals, including mucosal ulcerations, transmural necrosis, hemorrhage, and multifocal infarctions.

FIG. 6A shows camostat antiviral activity against SARS-CoV-2 in Calu-3 cells.

FIG. 6B shows relacatib antiviral activity against SARS-CoV-2 in Calu-3 cells.

FIG. 7A shows SARS-CoV-2 antiviral activity in primary human lung cells cultured in air liquid interface by varying concentrations of camostat and relacatib, compared to remdesivir main plasma metabolite.

FIG. 7B shows SARS-CoV-2 antiviral activity in primary human lung cells cultured in air liquid interface (ALI) by varying concentrations of camostat and relacatib, compared to remdesivir main plasma metabolite, specifically at day 6.

FIG. 8A shows primary human lung cells cultured in air liquid interface (ALI) efficacy data for relacatib (4 μM) and camostat (30 μM) combined, compared to relacatib alone, camostat alone and remdesivir main plasma metabolite.

FIG. 8B shows primary human lung cells cultured in air liquid interface (ALI) cytoxicity data (no virus) for relacatib (4 μM) and camostat (30 μM) combined, compared to relacatib alone, camostat alone and remdesivir main plasma metabolite.

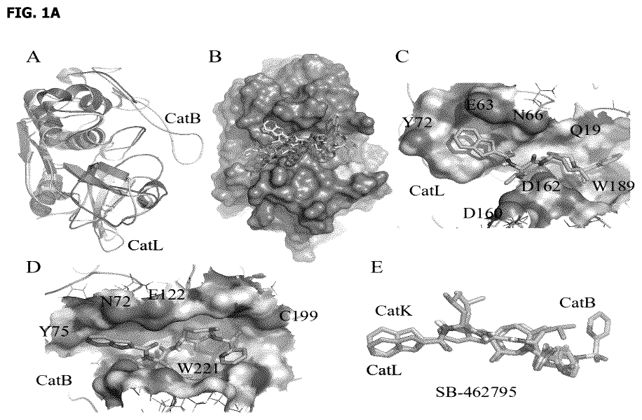

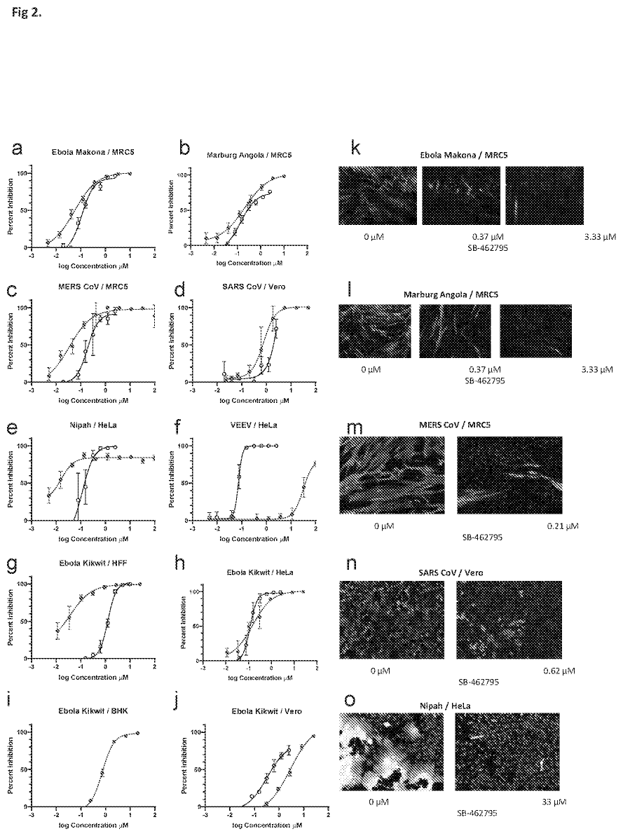

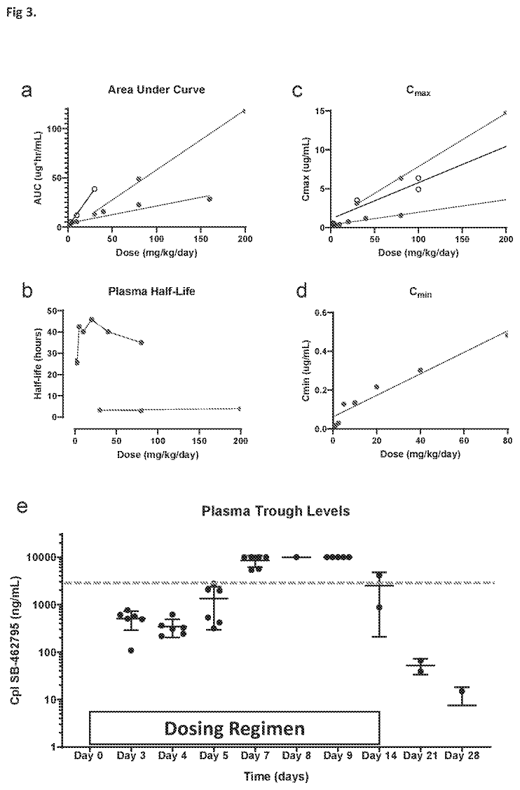

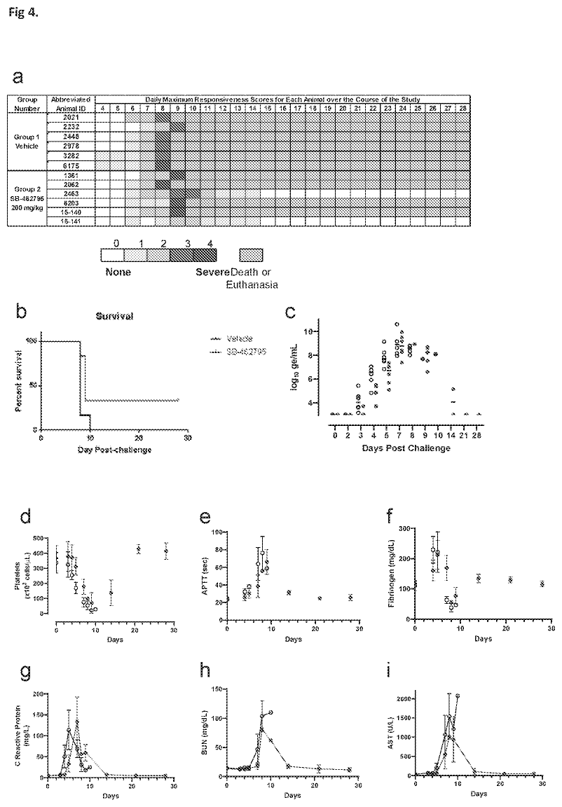

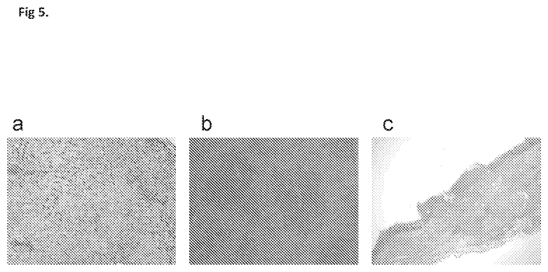

### DETAILED DESCRIPTION OF REPRESENTATIVE EMBODIMENTS

An object of the present invention is to provide compounds which inhibit cysteine proteases, more particularly compounds which inhibit cysteine proteases of the papain superfamily, yet more particularly compounds which inhibit cysteine proteases of the cathepsin family, for use or a method of treatment or prophylaxis of a viral disease in a subject caused by a virus, the method comprising administering such compound or a pharmaceutically acceptable salt thereof, to the subject in need thereof. Particular embodiments of the invention provide compounds that inhibit any of cathepsin K (Cat K), cathepsin L (Cat L), cathepsin V (Cat V (L2)), cathepsin S (Cat S), cathepsin G (Cat G), cathepsin D (Cat D), Calpain and/or Caspase 3-4, for use or a method of treatment or prophylaxis of a viral disease in a subject caused by a virus, the method comprising administering to the subject in need thereof a therapeutically effective amount of a compound as described herein, particularly SB-462795, or a pharmaceutically acceptable salt thereof.

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings.

As used herein unless otherwise specified, “alkyl” refers to a monovalent saturated aliphatic hydrocarbyl group having from 1 to 8 carbon atoms and, in some embodiments from 1 to 8 carbons, and in some embodiments from 1 to 6 carbon atoms. “(Cₓ-C_{y})alkyl” refers to alkyl groups having from x to y carbon atoms. The term “alkyl” includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Close contacts” and “Close contacts of a subject infected with SARS-CoV-2” are defined as (i) persons living in the same household as the infected subject; (ii) persons having had direct or physical contact with the infected subject; (iii) persons having remained within two metres of an infected subject for longer than 15 minutes on or after the date on which symptoms were first reported by the subject, or for an asymptomatic subject, in the 2 days prior to test specimen collection.

“Compound”, “compounds”, “chemical entity”, and “chemical entities” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“COVID-19” refers to the collection of symptoms (e.g. https://www.cdc.gov/coronavirus/2019-ncov/symptoms-testing/symptoms.html) exhibited by patients infected with any strain or Glade of SARS-CoV-2. Symptoms typically include cough, fever and shortness of breath (dyspnoea), although some patients are asymptomatic, in which case COVID-19 refers to SARS-CoV-2 infection alone.

“Secondary condition associated with COVID-19” or “secondary conditions associated with COVID-19” means any one or more of a myriad of symptoms associated with COVID-19 including but not limited to fever; chills; cough; shortness of breath; difficulty breathing; fatigue; muscle ache; body ache; headache; chest pain; pink eye (conjunctivitis); rash; loss of taste; loss of smell; sore throat; congestion; runny nose; nausea; vomiting; diarrhea; heart palpitations; racing heartbeat; Takotsubo cardiomyopathy; light-headedness; feeling faint; brain fogginess; numbness in fingers, hands, feet, limbs; tingling sensation in the body; postural orthostatic tachycardia syndrome (POTS); less effective blood pumping; inflammation of the heart; inflammation of the membrane around the heart; blood clots; and neurological symptoms.

“Cytokine Storm”, as used herein refers to excessive and/or uncontrolled release of proinflammatory cytokines, such as what has been observed to occur in a wide variety of infectious and non-infectious diseases, and particularly in viral disease, where cellular and molecular mechanisms may both be contributing to the cytokine storm seen in such disease, e.g. coronavirus diseases such as SARS, COVID-19, MERS, and influenza, Ebola and other viral diseases. Cytokine storm, as used herein, refers to the excessive immune response described by, for example, Tisoncik, J. R. et al. in Microbiol. Mol. Blot Rev. (2012), 76(1), 16-32 (“Into the Eye of the Cytokine Storm”).

“High risk” subjects and “high risk” categories include the following: subjects of 60 years of age and over; subjects with a high body-mass index (BMI)≥35; smokers, subjects having a chronic medical condition including heart disease, lung disease, diabetes, cancer or high blood pressure; immunocompromised subjects such as subjects undergoing treatment for cancer or autoimmune diseases such as rheumatoid arthritis, systemic lupus erythematosus, multiple sclerosis and inflammatory bowel disease, subjects having a transplant and HIV positive individuals, including persons living in a community residence such as a dormatory, assisted living facility, nursing home, rehabilitation center and the like, persons having attended a gathering of 10, 25, 50, 100, 500, 1000 or more people not separated by ˜6-feet (2 meters) and/or not protected by a face mask or shield, and persons having travelled to, from or through a region with high levels of SARS-CoV-2 and COVID-19 cases.

“Pharmaceutically acceptable salt” refers to pharmaceutically acceptable salts derived from a variety of organic and inorganic counter ions and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002. Other suitable pharmaceutically acceptable salts are described herein.

“Patient” or “subject” refers to animals, particularly mammals, and includes humans and non-human mammals.

“Prevention of COVID-19” is interpreted in accordance with the usual meaning of the word “prevent”.

“Racemates” refers to a mixture of enantiomers. In an embodiment of the invention, the compounds described herein, or pharmaceutically acceptable salts thereof, are enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

“SARS-CoV-2” is a beta coronavirus having greater than 90% sequence identity at the RNA level with any one of the sequences deposited in the China National Microbiological Data Centre under accession number NMDC10013002, or greater than 90% sequence identity at the RNA level with any one of the sequences deposited in the Global Initiative on Sharing All Influenza Data (GISAID) under reference NC_045512.2 SARS-CoV-2 Wuhan genome (Coronaviridae Study Group of the International Committee on Taxonomy of Viruses, 2020). In another embodiment, the SARS-CoV-2 coronavirus has greater than 95% sequence identity at the RNA level with any one of the sequences deposited in the China National Microbiological Data Centre under accession number NMDC10013002 or with reference NC_045512.2 SARS-CoV-2 Wuhan genome (GISAID). In other embodiments, the SARS-CoV-2 coronavirus has greater than 96% sequence identity, greater than 97% sequence identity, greater than 98% sequence identity or greater than 99% sequence identity at the RNA level with any one of the sequences deposited in the China National Microbiological Data Centre under accession number NMDC10013002 or with reference NC_045512.2 SARS-CoV-2 Wuhan genome (GISAID). The definition of SARS-CoV-2 is intended to cover all strains of SARS-CoV-2 including the L, S, G, GH, GR, V and O clades, as well as recent variants thereof, including the UK variant (201/501Y.V1/B.1.1.7), the South Africa variant (20H/501Y.V2/B.1.351), the Brazil variant (P.1/20J/501Y.V3/B.1.1.248) and the novel California variant descended from cluster 20C, defined by 5 mutations (ORF1a:I4205V, ORF1b:D1183Y, S:S13I; W152C; L452R) and designated CAL.20C (20C/S;452R;B.1.429). As defined, S Glade has a T at position 8782 and a C at position 28144; L Glade has a C at position 8782 and a T at position 28144; G Glade has a G at position 23403 (A23403G); GH Glade has a T at position 25563 (G25563T); GR Glade has a AAC for GGG starting at position 28881 (GGG28881AAC); Glade V has a T at position 26144 (ORG2a:G251V); and O has sequence variations and mutations not defined by clades L, S, G, GH, GR or V, with numbering relating to the reference genome of 2019-nCoV-2 (NC_045512). Of note, the actual RNA base in the SARS-CoV-2 genome is U—uracil—but to be consistent with the original NCBI NC_045512.2 reference genomic notation, T is used here to characterize the genetic events. The definition of SARS-CoV-2 also encompasses SARS-CoV-2 clades that have amino acid changes for Glade S (ORF8:L84S mutation), Glade G (S:D614G mutation), Glade GH (ORF3a:Q57H mutation), Glade GR (both S:D614G and N:RG203KR mutations), Glade V (ORF3a:G251V mutation), and Glade O (sequences and mutations not matching any of these criteria for the other clades), as well as other emerging variants descended from the clades defined above.

“Solvate” or “solvates” of a compound refer to those compounds, as defined above, which are bound to a stoichiometric or non-stoichiometric amount of a solvent. Solvates of a compound includes solvates of all forms of the compound. In certain embodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Therapeutically effective amount” or as used herein mean refers to an amount that is effective to elicit the desired biological or medical response, including the amount of a compound that, when administered to a subject for treating a disease, is sufficient to effect such treatment for the disease. The effective amount will vary depending on the particular compound and characteristics of the subject to be treated, such as age, weight. etc. The effective amount can include a range of amounts. As is understood in the art, a therapeutically effective amount may be in one or more doses, i.e., a single dose or multiple doses may be required to achieve the desired treatment endpoint. A therapeutically effective amount may be considered in the context of administering one or more therapeutic agents, and a single agent may be considered to be given in a therapeutically effective amount if, in conjunction with one or more other agents, a desirable or beneficial result may be or is achieved. Suitable doses of any co-administered compounds may optionally be lowered due to the combined action (e.g., additive or synergistic effects) or the compounds.

“Treating or “treatment or prophylaxis of a disease in a subject” refers to 1) preventing the disease from occurring in a subject that is predisposed or does not yet display symptoms of the disease; 2) inhibiting the disease or arresting its development; or 3) ameliorating or causing regression of the disease.

“Treatment of COVID-19” refers to a reduction in the viral load of SARS-CoV-2 and/or to a reduction in the viral titre of SARS-CoV-2, and/or to a reduction in the severity or duration of the symptoms of the disease. Viral load may be measured by a suitable quantitative RT-PCR assay or a suitable qualitative diagnostic test such as a RDT based on LFIA performed on a specimen from the patient. In one embodiment, the specimen may be a specimen from the upper or lower respiratory tract (such as a nasopharyneal or oropharyngeal swab, sputum, lower respiratory tract aspirates, bronchoalveolar lavage, bronchial biopsy, transbronchial biopsy and nasopharyngeal wash/aspirate or nasal aspirate) saliva or plasma. In a particular embodiment the specimen is mucous. In a more particular embodiment, the specimen is saliva. The protocols of a number of quantitative RT-PCR assays are published on https://www.who.int/emergencies/diseases/novel-coronavirus-2019/technical-guidance/laboratory-guidance. In addition, Corman and colleagues have published primers and probes for use in such assays (Corman et al., European communicable disease bulletin, 2020, DOI:10.2807/1560-7917). In one embodiment, the COVID-19 RdRp/He1 assay is used. This has been validated with clinical specimens and has a limit of detection of 1.8 TCID50/mL with genomic RNA and 11.2 RNA copies/reaction with in vitro RNA transcripts. (Chan et al., J Clin Microbiol., 2020, doi:10.1128/JCM.00310-20). Viral titre may be measured by assays well known in the art.

In one embodiment, treatment of COVID-19 refers to at least a 5 fold, 10 fold, 50 fold, 100 fold, 500 fold or 1000 fold reduction in the viral load (RNA copies/ml) measured by the same assay from a specimen from the same origin taken prior to treatment (baseline) and the end of the treatment period in a single patient. In one embodiment, treatment of COVID-19 refers to a greater than 0.5 log unit reduction in viral load. In another embodiment, treatment of COVID-19 refers to the situation where the mean viral load (RNA copies/ml) from specimens of the same origin from 30 patients measured in the same assay being reduced by at least 5 fold, 10 fold, 50 fold, 100 fold, 500 fold or 1000 fold at the end of the treatment period compared to baseline.

In one embodiment, treatment of COVID-19 refers to clinical improvement in signs and symptoms, as evidenced by patient vitals, patient self-reporting, and clinical observations.

In one embodiment, treatment of COVID-19 refers to the viral load being decreased to below the limit of detection of the 19 RdRp/He1 assay at the end of the treatment period.

Where specific compounds or generic formulas are drawn that have aromatic rings, such as aryl or heteroaryl rings, it will be understood by one of still in the art that the particular aromatic location of any double bonds are a blend of equivalent positions even if they are drawn in different locations from compound to compound or from formula to formula. For example, in the two pyridine rings (A and B) below, the double bonds are drawn in different locations, however, they are known to be the same structure and compound:

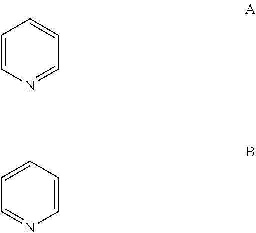 [SMILES: c1ccncc1 ; c1ccncc1]

The present invention is directed to compounds, compositions and pharmaceutical compositions that are serine protease inhibitors, more particularly cathepsin inhibitors, and have utility as treatments and/or preventative therapies for viral disease in a subject caused by a virus.

Viruses are classified by evaluating several characteristics, including the type of viral genome. Viral genomes can be comprised of DNA or RNA, can be double-stranded or single-stranded (which can further be positive-sense or negative-sense), and can vary greatly by size and genomic organization. An RNA virus is a virus that has RNA (ribonucleic acid) as its genetic material. This nucleic acid is usually single-stranded RNA (ssRNA). RNA viruses can be further classified according to the sense or polarity of their RNA into negative-sense and positive-sense. Positive-sense viral RNA is similar to mRNA and thus can be immediately translated by the host cell. Negative-sense viral RNA is complementary to mRNA and thus must be converted to positive-sense RNA by an RNA polymerase before translation. As such, purified RNA of a positive-sense virus can directly cause infection though it may be less infectious than the whole virus particle. Purified RNA of a negative-sense virus is not infectious by itself, as it needs to be transcribed into positive-sense RNA; each virion can be transcribed to several positive-sense RNAs.

Positive-sense, single-stranded RNA viruses (“positive-stand RNA viruses”) make up a large super family of viruses from many distinct subfamilies. These viruses span both the plant and animal kingdoms causing pathologies ranging from mild phenotypes to severe debilitating disease. The composition of the positive strand RNA virus polymerase supergroup includes, at least, the following families: levi-, narna-, picorna-, dicistro-, marna-, sequi-, como-, poty-, calici-, astro-, noda-, tetra-, luteo-, tombus-, corona-, arteri-, roni-, flavi-, toga-, bromo-, tymo-, clostero-, flexi-, seco-, barna, ifla-, sadwa-, chera-, hepe-, sobemo-, umbra-, tobamo-, tobra-, hordei-, furo-, pomo-, peclu-, beny-, ourmia-, and idaeovirus.

Negative-sense, single-stranded RNA viruses (“negative-strand RNA viruses”) make up a smaller super family of viruses from distinct subfamilies. These viruses also cause pathologies ranging from mild phenotypes to severe debilitating disease and death. The composition of the negative-strand RNA virus polymerase supergroup includes, at least, the following families: Bornaviridae, Filoviridae, Paramyxoviridae, Rhabdoviridae, Nyamiviridae, Arenaviridae, Bunyaviridae, Ophioviridae, Orthomyxoviridae, Deltavirus, Dichorhavirus, Emaravirus, Nyavirus, and Varicosavirus.

### Identification of Subjects Infected with SARS-CoV-2

Subjects infected with SARS-CoV-2 may be identified by detection of viral RNA from SARS-CoV-2 from a specimen obtained from the subject. Without intending to be limiting, the specimen may be a specimen from the upper or lower respiratory tract (such as a nasopharyneal or oropharyngeal swab, sputum, lower respiratory tract aspirates, bronchoalveolar lavage and nasopharyngeal wash/spirate or nasal aspirate). Any known methods of RNA detection may be used, such as high-throughput sequencing or real-time reverse-transcriptase polymerase-chain-reaction (RT-PCR) assay. In one embodiment, the method comprises the following steps:
Isolating RNA from a specimen;
Reverse transcription of the RNA;
Amplification with forward and reverse primers in the presence of a probe; and
Detection of the probe;
   wherein the presence of SARS-CoV-2 is confirmed if the cycle threshold growth curves cross the threshold within 40 cycles.

In a more particular embodiment, step c) utilises the following:

Fwd Primer
(SEQ ID NO: 1)
5′ GACCCCAAAATCAGCGAAAT 3′
Rev Primer
(SEQ ID NO: 2)
5′ TCTGGTTACTGCCAGTTGAATCTG 3′
Probe
(SEQ ID NO: 3)
5′ FAM-ACCCCGCATTACGTTTGGTGGACC-BHQ-1 3′

In an alternative embodiment, step c) utilises the following:

Fwd Primer
(SEQ ID NO: 4)
5′ TTACAAACATTGGCCGCAAA 3′
Rev Primer
(SEQ ID NO: 5)
5′ GCGCGACATTCCGAAGAA 3′
Probe
(SEQ ID NO: 6)
5′ FAM-ACAATTTGCCCCCAGCGCTTCAG-BHQ-1 3′

These primers and probes are commercially available from Integrated DNA Technologies (Catalogue No. 10006606) and BioSearch Technologies (Catalogue No. KIT-nCoV-PP1-1000). Detailed instructions for performing real-time reverse-transcriptase polymerase-chain-reaction (RT-PCR) assay using these primers has been published by the CDC (https://www.cdc.gov/coronavirus/2019-nCoV/lab/index.html).

Accordingly, in one embodiment, the invention comprises a method for treating COVID-19 in a subject comprising a method of detecting viral RNA from SARS-CoV-2 from a specimen obtained from the subject and, where viral RNA is detected, a step of treating COVID-19 as described herein.

In one aspect, the invention provides a method for testing for SARS-CoV-2 in a subject and treating SARS-CoV-2 infection in the subject, which method comprises the following steps:
Isolating RNA from a specimen derived from a subject;
Reverse transcription of the RNA;
Amplification with forward and reverse primers in the presence of a probe; and
Detection of the probe;
   wherein the subject is defined as having SARS-CoV-2 infection if the cycle threshold growth curves cross the threshold within 40 cycles; and
treating the subject having SARS-CoV-2 infection with a therapeutically effective amount of a compound which is:
N-((2S)-4-methyl-1-(((4S,7R)-7-methyl-3-oxo-1-(pyridin-2-ylsulfonyl)azepan-4-yl)amino)-1-oxopentan-2-yl)benzofuran-2-carboxamide;
3-methyl-N-((S)-4-methyl-1-(((4S,7R)-7-methyl-3-oxo-1-(pyridin-2-ylsulfonyl)azepan-4-yl)amino)-1-oxopentan-2-yl)furo[3,2-b]pyridine-2- carboxamide,
   or a pharmaceutically acceptable salt thereof.

In a particular embodiment, the compound is.

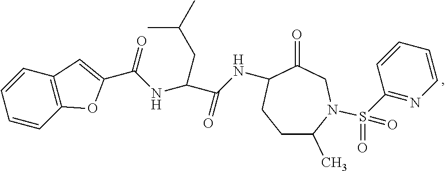 [SMILES: CC(C)CC(NC(=O)c1cc2ccccc2o1)C(=O)NC1CCC(C)N(S(=O)(=O)c2ccccn2)CC1=O]

or a pharmaceutically acceptable salt thereof.

In specific embodiments of this method, the subject is human, and the specimen and/or the primers and probe are as described above. The treatment may also be conducted as described herein.

In some embodiments, the method of identification of subjects infected with SARS-CoV-2 is capable of identifying whether the subject is infected with L strain SARS-CoV-2 RNA, S strain SARS-CoV-2 RNA, G strain SARS-CoV-2 RNA, GH strain SARS-CoV-2 RNA, GR strain SARS-CoV-2 RNA, V strain SARS-CoV-2 RNA, or O strain SARS-CoV-2 RNA. The method described herein could include a further step of sequencing amplified cDNA to identify whether the subject is infected with S strain, L strain, G strain, GH strain, GR strain, V strain or O strain, as well as a further step to identify whether the subject is infected with a recent variant thereof, including the UK variant (201/501Y.V1/B.1.1.7), the South Africa variant (20H/501Y.V2/B.1.351), the Brazil variant (P.1/20J/501Y.V3/13.1.1.248) and the novel California variant descended from cluster 20C, defined by 5 mutations (ORF1a:I4205V, ORF1b:D1183Y, S:S13I; W152C; L452R) and designated CAL.20C (20C/S;452R;B.1.429).

In an alternative embodiment, subjects infected with SARS-CoV-2 may be identified by detection of an SARS-CoV-2 antigen or subject antibodies directed to SARS-CoV-2 in a sample of blood or mucous taken from the subject. In one embodiment, subjects infected with SARS-CoV-2 may be identified by detection of an SARS-CoV-2 antigens in a sample of blood or mucous taken from the subject. Any suitable assay may be used. Kits for conducting such serological assays are already commercially available, e.g. from Biomerica and Pharmact. Details of performance of authorised serology tests is available on https://www.fda.gov/medical-devices/coronavirus-disease-2019-covid-19-emergency-use-authorizations-medical-devices/eua-authorized-serology-test-performance.

In one embodiment, the assay to identify subjects infected with SARS-CoV-2 comprises:
a) contacting at least one immobilised antigen from SARS-CoV-2 with blood from the subject; and
b) detection of a complex formed between subject antibodies directed to the immobilised antigen and the immobilised antigen;
   where the subject is identified to be infected with SARS-CoV-2 if a complex is detected in step b).

In a particular embodiment of this assay, the antigen from SARS-CoV-2 is selected from the N-protein and the S protein or fragments thereof. In a more particular embodiment, the antigen from SARS-CoV-2 is selected from the N-protein, the S1 domain of the S protein and the S2 domain of the S protein. In one embodiment, the assay comprises more than one immobilised antigen.

In one embodiment, there is a step of washing the immobilised antigen after step a) and before step b).

In one embodiment, the detection step b) comprises contacting the complex formed with a labelled antibody or antibodies recognising the same antigen or antigens followed by detection of the label. In a more particular embodiment, the complex is washed after addition of labelled antibody(ies) prior to detection of the label.

In one embodiment, the label is capable of producing a coloured product, enabling visual detection of the label.

In one embodiment, the assay is a lateral flow assay. In more particular embodiment, the lateral flow assay has the immobilised antigen(s) on a dipstick.

In one embodiment, the invention provides a method for testing for SARS-CoV-2 in a subject and treating SARS-CoV-2 infection in the subject, which method comprises the following steps:
a) contacting at least one immobilised antigen from SARS-CoV-2 with blood from the subject; and
b) detecting a complex formed between subject antibodies directed to the immobilised antigen and the immobilised antigen;
   where the subject is identified to be infected with SARS-CoV-2 if a complex is detected in step b); and
c) treating the subject having SARS-CoV-2 infection with a therapeutically effective amount of a compound selected from:
N-((2S)-4-methyl-1-(((4S,7R)-7-methyl-3-oxo-1-(pyridin-2-ylsulfonyl)azepan-4-yl)amino)-1-oxopentan-2-yl)benzofuran-2-carboxamide;
3-methyl-N-((S)-4-methyl-1-(((4S,7R)-7-methyl-3-oxo-1-(pyridin-2-ylsulfonyl)azepan-4-yl)amino)-1-oxopentan-2-ypfuro[3,2-b]pyridine-2- carboxamide,
   or a pharmaceutically acceptable salt thereof.

In a particular embodiment, the compound is

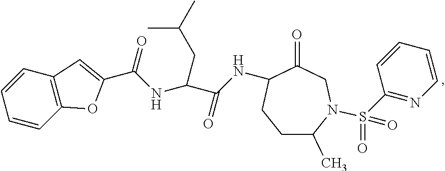 [SMILES: CC(C)CC(NC(=O)c1cc2ccccc2o1)C(=O)NC1CCC(C)N(S(=O)(=O)c2ccccn2)CC1=O]

or a pharmaceutically acceptable salt thereof. In specific embodiments of this method, the subject is human, and the assay is conducted as described above. The treatment may also be conducted as described herein.

In one embodiment, the assay to identify subjects infected with SARS-CoV-2 comprises:
a) contacting an immobilised antibody recognising an antigen from SARS-CoV-2 with blood from the subject; and
b) detection of a complex formed between an antigen from SARS-CoV-2 and the immobilised antibody recognising said antigen;
   where the subject is identified to be infected with SARS-CoV-2 if a complex is detected in step b).

In a particular embodiment of this assay, the antigen from SARS-CoV-2 is selected from the N-protein and the S protein or fragments thereof. In a more particular embodiment, the antigen from SARS-CoV-2 is selected from the N-protein, the S1 domain of the S protein and the S2 domain of the S protein. In one embodiment, the assay comprises more than one immobilised antibody, each antibody recognising a different antigen.

In one embodiment, there is a step of washing the immobilised antibody after step a) and before step b).

In one embodiment, the detection step b) comprises contacting the complex formed in step a) with labelled antibodies recognising the same antigen or antigens followed by detection of the label. In a more particular embodiment, step b) comprises a step of washing prior to detection of the label.

In one embodiment, the label is capable of producing a coloured product, enabling visual detection of the label.

In one embodiment, the assay is a lateral flow assay. In more particular embodiment, the lateral flow assay has the immobilised antibod(ies) on a dipstick.

In one embodiment, the invention provides a method for testing for and treating SARS-CoV-2 infection, which method comprises the following steps:
a) contacting an immobilised antibody recognising an antigen from SARS-CoV-2 with blood from the subject; and
b) detecting of a complex formed between an antigen from SARS-CoV-2 and the immobilised antibody recognising said antigen;
   wherein the subject is identified to be infected with SARS-CoV-2 if a complex is detected in step b), and treating the subject having SARS-CoV-2 infection with a therapeutically effective amount of a compound selected from:
N-((2S)-4-methyl-1-(((4S,7R)-7-methyl-3-oxo-1-(pyridin-2-ylsulfonyl)azepan-4-yl)amino)-1-oxopentan-2-yl)benzofuran-2-carboxamide;
3-methyl-N-((S)-4-methyl-1-(((4S,7R)-7-methyl-3-oxo-1-(pyridin-2-ylsulfonyl)azepan-4-yl)amino)-1-oxopentan-2-yl)furo[3,2-b]pyridine-2- carboxamide,
   or a pharmaceutically acceptable salt thereof.

In a particular embodiment, the compound is N-((2S)-4-methyl-1-(((4S,7R)-7-methyl-3-oxo-1-(pyridin-2-ylsulfonyl)azepan-4-yl)amino)-1-oxopentan-2-yl)benzofuran-2-carboxamide

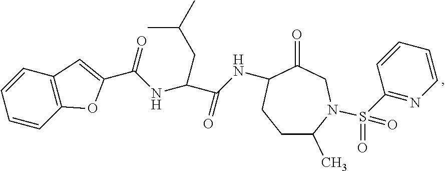 [SMILES: CC(C)CC(NC(=O)c1cc2ccccc2o1)C(=O)NC1CCC(C)N(S(=O)(=O)c2ccccn2)CC1=O]

or a pharmaceutically acceptable salt thereof. In specific embodiments of this method, the subject is human, and the assay is conducted as described above. The treatment may also be conducted as described herein.

One aspect of the invention provides a method for the treatment or prophylaxis of a viral disease in a subject in need thereof, the method comprising administering to the subject a therapeutically effective amount of a compound of the structure:

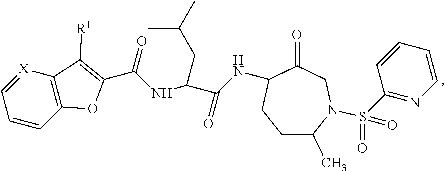

or a pharmaceutically acceptable salt thereof,
wherein:
X is selected from N or CR²; and
R¹ and R² is each independently selected from —H or —(C₁-C₈)alkyl.

Particular embodiments provide a method for the treatment or prophylaxis of a viral disease in a subject in need thereof as described, wherein R¹ is —(C₁-C₈)alkyl.

Particular embodiments provide a method for the treatment or prophylaxis of a viral disease in a subject in need thereof as described, wherein R² is H.

Particular embodiments provide a method for the treatment or prophylaxis of a viral disease in a subject in need thereof as described, wherein R¹ is —CH₃ and R² is H.

One particular embodiment provides a method for the treatment or prophylaxis of a viral disease in a subject in need thereof, the method comprising administering to the subject a therapeutically effective amount of a compound as described, wherein the compound is:

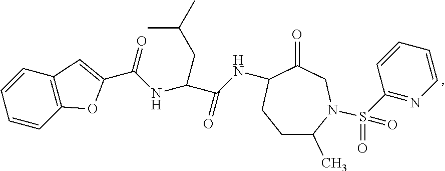 [SMILES: CC(C)CC(NC(=O)c1cc2ccccc2o1)C(=O)NC1CCC(C)N(S(=O)(=O)c2ccccn2)CC1=O]

or a pharmaceutically acceptable salt thereof.

One particular embodiment provides a method for the treatment or prophylaxis of a viral disease in a subject in need thereof, the method comprising administering to the subject a therapeutically effective amount of a compound as described, wherein the compound is:

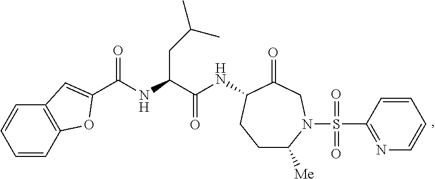 [SMILES: CC(C)C[C@H](NC(=O)c1cc2ccccc2o1)C(=O)N[C@H]1CC[C@@H](C)N(S(=O)(=O)c2ccccn2)CC1=O]

or a pharmaceutically acceptable salt thereof.

Related embodiments provide a method for the treatment or prophylaxis of a viral disease in a subject in need thereof, the method comprising administering to the subject a therapeutically effective amount of a first therapeutic agent that as described herein, particularly SB-462795 or a pharmaceutically acceptable salt thereof, in combination with a therapeutically effective amount of a second therapeutic agent, wherein the second therapeutic agent is not the first therapeutic agent.

In related embodiments, the second therapeutic agent is an agent that blocks viral entry into a cell of the animal, an agent that acts directly on the virus, such as by modulating a viral protein, an agent that inhibits the virus by modulating a protein, receptor or target in the animal other than a cathepsin, an agent that modulates furin and related proprotein convertase (PC) proteases (see Cheng et al., Furin Inhibitors Block SARS-CoV-2 Spike Protein Cleavage to Suppress Virus Production and Cytopathic Effects, Cell Reports, 33, pp. 108254, 2020) e.g. peptide inhibitors such as decanoyl-RVKR-chloromethylketone (CMK) or small molecule inhibitors such as naphthofluorescein, an agent that modulates transmembrane protease serine 2 (TMPRSS2) e.g. camostat, nafamostat or a pharmaceutically acceptable salt thereof, an agent that modulates a receptor in the animal, e.g. an angiotensin-converting enzyme 2 (ACE 2) inhibitor, an immunomodulator and prevents or ameliorates a cytokine storm in the subject, e.g. a 4-aminoquinoline compound or an 8-aminoquinoline compound including chloroquine, hydroxychloroquine, tafenoquine, primaquine, or a pharmaceutically acceptable salt thereof, in particular wherein the subject is an animal, more particularly a human.

Particular embodiments provide a method for the treatment or prophylaxis of a viral disease in a subject in need thereof, the method comprising administering to the subject a therapeutically effective amount of a first therapeutic agent that as described herein, particularly SB-462795 or a pharmaceutically acceptable salt thereof, wherein the viral disease is caused by a negative-strand RNA virus or a positive-strand RNA virus.

Particular embodiments provide a method for the treatment or prophylaxis of a viral disease in a subject in need thereof, the method comprising administering to the subject a therapeutically effective amount of a first therapeutic agent that as described herein, particularly SB-462795 or a pharmaceutically acceptable salt thereof, wherein the viral disease is caused by virus selected from the group consisting of picornaviridae family, flaviviridae family, filoviridae family, and coronaviridae family.

Particular embodiments provide a method for the treatment or prophylaxis of a viral disease in a subject in need thereof, the method comprising administering to the subject a therapeutically effective amount of a first therapeutic agent that as described herein, particularly SB-462795 or a pharmaceutically acceptable salt thereof, wherein the viral disease is caused by a poliovirus, a rhinovirus, a coxsackievirus (a foot-and-mouth virus (FMDV)), an influenza virus (influenza A virus, influenza B virus, influenza C virus, an avian influenza virus), a flavivirus, a hepatitis virus (hepatitis A virus, hepatitis B virus, hepatitis C virus, hepatitis D virus, hepatitis E virus), a coronavirus (SARS-CoV, SARS-CoV-2, a coronavirus responsible for Middle Eastern Respiratory Syndrome (MERS)), an Ebola virus, a Marburg virus, a Nipah virus (NiV), a Hendra virus, an arenavirus, a Rift Valley Fever virus, a yellow fever virus, a respiratory syncytial virus (RSV), a West Nile virus, a Dengue fever virus, an Aichi virus, an enterovirus, a rubella virus, a Theiler's murine encephalomyelitis virus (TMEV), an echovirus, or a human immunodeficiency virus (HIV).

In related embodiments there is provided a method for the treatment or prophylaxis of a viral disease in a subject in need thereof, the method comprising administering to the subject a therapeutically effective amount of a first therapeutic agent that as described herein, particularly SB-462795 or a pharmaceutically acceptable salt thereof, wherein the virus is a coronavirus, particularly SARS-CoV, more particularly SARS-CoV-2.

In related embodiments there is provided a method for the treatment or prophylaxis of a viral disease in a subject in need thereof, the method comprising administering to the subject a therapeutically effective amount of a first therapeutic agent that as described herein, particularly SB-462795 or a pharmaceutically acceptable salt thereof, wherein the viral disease is COVID-19, Severe Acute Respiratory Syndrome (SARS), or (MERS).

Particular embodiments provide a compound of the structure:

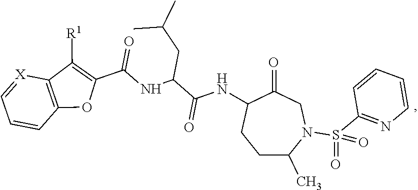

or a pharmaceutically acceptable salt thereof,
wherein:
X is selected from N or CR²; and
R¹ and R² is each independently selected from —H or —(C₁-C₈)alkyl, for use in antiviral therapy.

Another embodiment provides a compound of the structure:

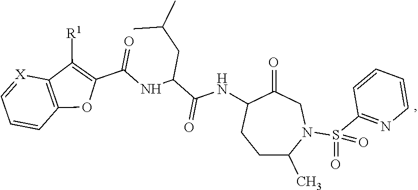

or a pharmaceutically acceptable salt thereof, for use in the treatment or prophylaxis of a viral disease,
wherein:
X is selected from N or CR²; and
R¹ and R² is each independently selected from —H or —(C₁-C₈)alkyl, for use in antiviral therapy.

In particular embodiments, the compound for use in the treatment or prophylaxis is as described, wherein R¹ is —(C₁-C₈)alkyl.

In particular embodiments, the compound for use in the treatment or prophylaxis is as described, wherein R² is H.

In particular embodiments, the compound for use in the treatment or prophylaxis is as described, wherein R¹ is —CH₃ and R² is H.

In particular embodiments, the compound for use in the treatment or prophylaxis is as described, wherein the compound is:

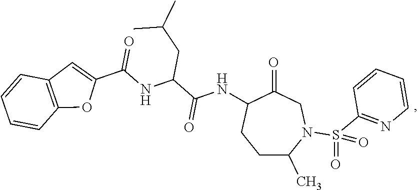 [SMILES: CC(C)CC(NC(=O)c1cc2ccccc2o1)C(=O)NC1CCC(C)N(S(=O)(=O)c2ccccn2)CC1=O]

wherein the virus is a poliovirus, a rhinovirus, a coxsackievirus, a foot-and-mouth virus (FMDV)), an influenza virus (influenza A virus, influenza B virus, influenza C virus, an avian influenza virus), a flavivirus, a hepatitis virus (hepatitis A virus, hepatitis B virus, hepatitis C virus, hepatitis D virus, hepatitis E virus), a coronavirus (CoV-SARS virus, coronavirus responsible for Middle Eastern Respiratory Syndrome (MERS), CoV-SARS-2), an Ebola virus, a Marburg virus, a Nipah virus (NiV), a Hendra virus, an arenavirus, a Rift Valley Fever virus, a yellow fever virus, a respiratory syncytial virus (RSV), a West Nile virus, a Dengue fever virus, an Aichi virus, an enterovirus, a rubella virus, a Theiler's murine encephalomyelitis virus (TMEV), an echovirus or a human immunodeficiency virus (HIV).

In particular embodiments, the compound for use in the treatment or prophylaxis is as described, wherein the compound is:

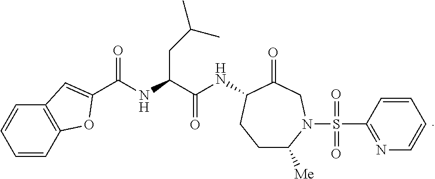 [SMILES: CC(C)C[C@H](NC(=O)c1cc2ccccc2o1)C(=O)N[C@H]1CC[C@@H](C)N(S(=O)(=O)c2ccccn2)CC1=O]

In particular embodiments, the compound for use in the treatment or prophylaxis is as described, wherein the compound is:

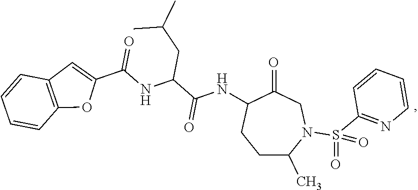 [SMILES: CC(C)CC(NC(=O)c1cc2ccccc2o1)C(=O)NC1CCC(C)N(S(=O)(=O)c2ccccn2)CC1=O]

for use as a first therapeutic agent, wherein the first therapeutic agent is administered in combination with a second therapeutic agent, wherein the second therapeutic agent is not the first therapeutic agent.

In related embodiments the compound for use as the first therapeutic agent in the treatment or nronhvlaxis of a viral disease is:

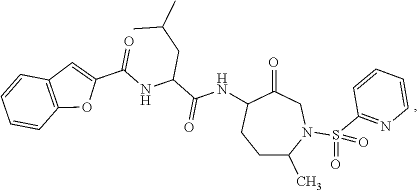 [SMILES: CC(C)CC(NC(=O)c1cc2ccccc2o1)C(=O)NC1CCC(C)N(S(=O)(=O)c2ccccn2)CC1=O]

and the second therapeutic agent is an agent that blocks viral entry into a cell of the animal, an agent that acts directly on the virus, such as by modulating a viral protein, an agent that inhibits the virus by modulating a protein, receptor or target in the animal other than a cathepsin, an agent that modulates furin and related proprotein convertase (PC) proteases e.g. peptide inhibitors such as decanoyl-RVKR-chloromethylketone (CMK) or small molecule inhibitors such as naphthofluorescein, an agent that modulates trypsin or trypsin-like proteases, an agent that modulates transmembrane protease serine 2 (TMPSS2), e.g. camostat, nafamostat or a pharmaceutically acceptable salt thereof, and agent that modulates a receptor in the animal, e.g. an angiotensin-converting enzyme 2 (ACE 2) inhibitor, an agent that acts as an immunomodulator and prevents or ameliorates a cytokine storm in the subject, e.g. an agent that acts as an immunomodulator is a 4-aminoquinoline compound or an 8-aminoquinoline compound, or a pharmaceutically acceptable salt thereof, including chloroquine, hydroxychloroquine, tafenoquine, primaquine or a therapeutically acceptable salt thereof.

In particular embodiments, the compound for use in the treatment or prophylaxis is as described, wherein the compound is:

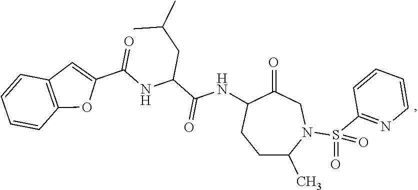 [SMILES: CC(C)CC(NC(=O)c1cc2ccccc2o1)C(=O)NC1CCC(C)N(S(=O)(=O)c2ccccn2)CC1=O]

and wherein the virus is a coronavirus, e.g. SARS-CoV or SARS-CoV-2.

In particular embodiments, the compound for use in the treatment or prophylaxis is as described, wherein the compound is:

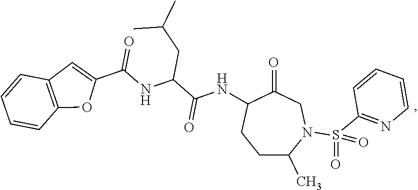 [SMILES: CC(C)CC(NC(=O)c1cc2ccccc2o1)C(=O)NC1CCC(C)N(S(=O)(=O)c2ccccn2)CC1=O]

wherein the viral disease is COVID-19, SARS or MERS.

In particular embodiments, the compound for use in the treatment or prophylaxis is as described, wherein the compound is:

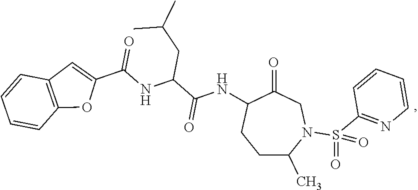 [SMILES: CC(C)CC(NC(=O)c1cc2ccccc2o1)C(=O)NC1CCC(C)N(S(=O)(=O)c2ccccn2)CC1=O]

wherein the virus is any of an Ebola virus, a Nipah virus, an enterovirus, an influenza virus, a human immunodeficiency virus, or a Zika virus wherein the viral disease is microencephaly, Guillain-Barré Syndrome, a severe brain abnormality, or a neurological disorder.

One embodiment provides use of a compound or a pharmaceutically acceptable salt thereof having the structure

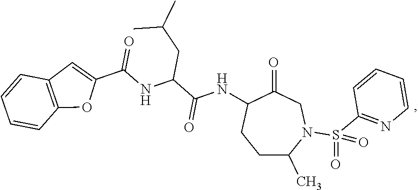 [SMILES: CC(C)CC(NC(=O)c1cc2ccccc2o1)C(=O)NC1CCC(C)N(S(=O)(=O)c2ccccn2)CC1=O]

in the manufacture of a medicament for use in treatment of a viral disease, wherein the virus is a poliovirus, a rhinovirus, a coxsackievirus, a foot-and-mouth virus (FMDV), an influenza virus (influenza A virus, influenza B virus, influenza C virus, an avian influenza virus), a flavivirus, a hepatitis virus (hepatitis A virus, hepatitis B virus, hepatitis C virus, hepatitis D virus, hepatitis E virus), a coronavirus (SARS-CoV, SARS-CoV-2, a coronavirus responsible for Middle Eastern Respiratory Syndrome (MERS) virus), an Ebola virus, a Marburg virus, a Nipah virus (NiV), a Hendra virus, an arenavirus, a Rift Valley Fever virus, a yellow fever virus, a respiratory syncytial virus (RSV), a West Nile virus, a Dengue fever virus, an Aichi virus, an enterovirus, a rubella virus, a Theiler's murine encephalomyelitis virus (TMEV), an echovirus, or a human immunodeficiency virus (HIV).

In related embodiments, there is provided use of a therapeutically effective amount of a first therapeutic agent that is the compound or pharmaceutically acceptable salt therein having the structure

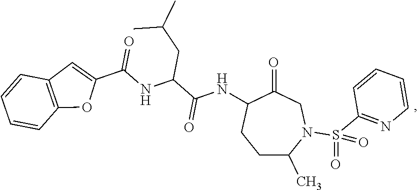 [SMILES: CC(C)CC(NC(=O)c1cc2ccccc2o1)C(=O)NC1CCC(C)N(S(=O)(=O)c2ccccn2)CC1=O]

in the manufacture of a medicament for use in treatment of a viral disease as described, in combination with a therapeutically effective amount of a second therapeutic, wherein the second therapeutic agent is a therapeutic agent is not the first therapeutic agent.

One embodiment provides use of a compound as described herein, particularly SB-462795, or a pharmaceutically acceptable salt thereof, in the manufacture of a medicament for use in treatment of a viral disease caused by a virus, wherein the virus is a coronavirus. More particularly, the coronavirus is SARS-CoV, SARS-CoV-2 or a coronavirus that causes MERS.

A particular embodiment provides use of a compound as described herein, particularly SB-462795, or a pharmaceutically acceptable salt thereof, in the manufacture of a medicament for use in treatment of a viral disease caused by a virus, wherein the virus is a coronavirus, and wherein the viral disease is SARS, COVID-19 or MERS.

One embodiment provides use of a compound as described herein, particularly SB-462795, or a pharmaceutically acceptable salt thereof, in the manufacture of a medicament for use in treatment of a viral disease caused by a virus, wherein the virus is a coronavirus wherein the virus is an Ebola virus.

One embodiment provides use of a compound as described herein, particularly SB-462795, or a pharmaceutically acceptable salt thereof, in the manufacture of a medicament for use in treatment of a viral disease caused by a virus, wherein the virus is a coronavirus, wherein the virus is a Zika virus.

In a particular embodiment there is provided use of a compound as described herein, particularly SB-462795, or a pharmaceutically acceptable salt thereof, in the manufacture of a medicament for use in treatment of a viral disease caused by a virus, wherein the virus is a coronavirus, wherein the virus is a Zika virus and the viral disease is microencephaly, Guillain-Barré Syndrome, a severe brain abnormality, or a neurological disorder.

One embodiment provides use of a compound as described herein, particularly SB-462795, or a pharmaceutically acceptable salt thereof, in the manufacture of a medicament for use in treatment of a viral disease caused by a virus, wherein the virus is a coronavirus, wherein the virus is a Nipah virus, an enterovirus, or an influenza virus.

In one embodiment there is provided a method for the treatment or prophylaxis of a viral disease in a subject caused by a virus, the method comprising administering to a subject in need thereof a therapeutically effective amount of a compound which is N-[(2S)-4-methyl-1-[[(4S,7R)-7-methyl-3-oxo-1-pyridin-2-ylsulfonylazepan-4-yl]amino]-1-oxopentan-2-yl]-1-benzofuran-2-carboxamide, or a pharmaceutically acceptable salt thereof, which is also known as SB-462795 or relacatib. Nomenclature is as provided at PubChem U.S. National Library of Medicine, National Center for Biotechnology Information for relacatib (https://pubchem.ncbi.nlm.nih.gov/compound/Relacatib).

In one embodiment of the present invention, the compounds described herein are useful for preventing or treating viral disease in a subject wherein the viral disease is caused by a single-stranded RNA virus.

In certain embodiments of the present invention, the compounds described herein are useful for preventing or treating viral disease in a subject wherein the viral disease is caused by a positive-sense, single-stranded RNA virus or the viral disease is caused by a negative-sense, single-stranded RNA virus.

In certain embodiments, there is provided a method for treating a viral disease in a subject in need thereof, wherein the viral disease is caused by a virus from a nidovirales, picornavirales, tymovirales, mononegavirales, reoviridae, pycobirnaviridae, parvoviridae, adenoviridae, poxviridae, polyomaviridae, herpesviridae, paramyxoviridae family of viruses, the method comprising administering to the subject a composition comprising any of the compounds described herein, particularly SB-462795, or a pharmaceutically acceptable salt thereof.

Embodiments provide compounds, methods and pharmaceutical compositions for treating viral disease wherein the viral disease is caused by a virus, the method comprising administering to a subject in need thereof a therapeutically effective amount of a compound as described herein, particularly SB-462795, or a pharmaceutically acceptable salt thereof. Methods for preparing such compounds and methods of using the compounds and pharmaceutical compositions thereof are also disclosed. In particular, embodiments for the treatment and prophylaxis of viral disease in a subject caused by an RNA or a DNA virus are disclosed.

In other embodiments, the compounds described herein, particularly SB-462795, are useful for treating a viral disease in a subject where the viral disease is caused by a virus belonging to the picornaviridae family, flaviviridae family, filoviridae family, paramyxoviridae family, or coronaviridae family. In other embodiments, the compounds described herein are useful for treating viral disease in a subject where the viral disease is caused by a virus belonging to the picornaviridae family. In other embodiments, the compounds described herein, particularly SB-462795, are useful for treating a viral disease in a subject where the viral disease is caused by a virus belonging to the coronaviridae family.

In yet other embodiments, the compounds described herein are useful for preventing or treating viral disease caused by a virus from any phylogenetic order, genus, family or particular species including, but not limited to, those listed in Table A below.

TABLE A
Positive-sense single stranded RNA viruses
Order Nidovirales
Family Arteriviridae
Family Coronaviridae - includes coronavirus, SARS-CoV, SARS-CoV-2
and coronavirus causing MERS
Family Roniviridae
Order Picornavirales
Family Bacillariornaviridae
Family Caliciviridae - includes Norwalk virus
Family Dicistroviridae
Family flaviviridae - includes Zika virus, dengue virus, yellow fever,
Japanese encephalitis, West Nile virus
Family Labyrnaviridae
Family Marnaviridae
Family Picornaviridae - includes enterovirus including poliovirus, the
“common cold” virus (rhinovirus), hepatitis A virus, coxsackievirus
Family Potyviridae
Family Secoviridae includes subfamily Comovirinae
Family Sequiviridae
Order Tymovirales
Family Alphaflexiviridae
Family Alphaflexiviridae
Family Betaflexiviridae
Family Gammaflexiviridae
Family Tymoviridae
Unassigned
Family Alvernaviridae
Family Astroviridae
Family Barnaviridae
Family Bromoviridae
Family Closteroviridae
Family Flaviviridae - includes Yellow fever virus, West Nile virus,
Hepatitis C virus, Dengue fever virus
Family Leviviridae
Family Luteoviridae
Family Narnaviridae
Family Nodaviridae
Family Retroviridae - includes human immunodeficiency virus 1 and 2
Family Tetraviridae
Family Togaviridae - includes Rubella virus, Ross River virus, Sindbis
virus, Chikungunya virus
Family Tombusviridae
Family Virgaviridae
Negative-sense single stranded RNA viruses
Order Mononegavirales
Family Bornaviridae - Borna disease virus
Family Filoviridae - includes Ebola virus, Marburg virus
Family Paramyxoviridae - includes Measles virus, Mumps virus, Nipah
virus, Hendra virus, respiratory syncytial virus (RSV), human
parainfluenza viruses (PIVs), human metapneumovirus (hMPV)
Family Rhabdoviridae - includes Rabies virus
Unassigned families:
Family Arenaviridae - includes Lassa virus, Junin virus
Family Bunyaviridae - includes Hantavirus, Crimean-Conghemorrhagic
fever
Family Ophioviridae
Family Orthomyxoviridae - includes Influenza viruses
Unassigned genera:
Genus Deltavirus
Genus Emaravirus
Genus Nyavirus - includes Nyamanini and Midway viruses
Double stranded RNA viruses
Family Reoviridae - includes Rotavirus
Family Pycobirnaviridae - includes human pycobirnavirus
DNA viruses
Family Parvoviridae - includes Parvovirus B19
Family Adenoviridae- includes adenovirus
Family Hepadenoviridae - includes hepatitis B, D, E
Family Poxviridae - includes monkey pox
Family Polyomaviridae - includes BK virus
Family Herpesviridae - includes herpes simplex virus

In further embodiments, the compound as described herein for method of treating or use, or a pharmaceutically acceptable salt thereof, is chosen from the compounds set forth in Table B below.

### Preparation of Compounds

TABLE B

| Compound | | |
|---|---|---|
| No. | Structure | Name |
| 1 | 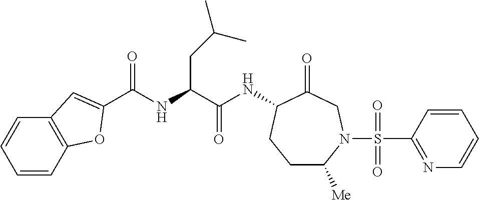 [SMILES: CC(C)C[C@H](NC(=O)c1cc2ccccc2o1)C(=O)N[C@H]1CC[C@@H](C)N(S(=O)(=O)c2ccccn2)CC1=O] | N-((2S)-4-methyl-1-(((4S,7R)-7- methyl-3-oxo-1-(pyridin-2- ylsulfonyl)azepan-4-yl)amino)-1- oxopentan-2-yl)benzofuran-2- carboxamide |
| 2 | 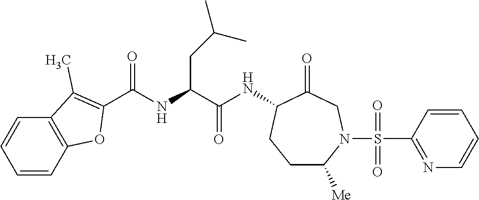 [SMILES: Cc1c(C(=O)N[C@@H](CC(C)C)C(=O)N[C@H]2CC[C@@H](C)N(S(=O)(=O)c3ccccn3)CC2=O)oc2ccccc12] | 3-methyl-N-((S)-4-methyl-1- (((4S,7R)-7-methyl-3-oxo-1- (pyridin-2-ylsulfonyl)azepan-4- yl)amino)-1-oxopentan-2- yl)furo[3,2-b]pyridine-2- carboxamide |

The compounds described herein and compounds 1 (SB-462795/relacatib) and 2 shown above, can be synthesized by one of skill in the art by reading the Synthetic Methods, General Schemes, and the Examples described in the following references, which are hereby incorporated by reference in their entireties: PCT published patent application No. WO 2001070232 to Cummings, et al., and PCT published patent application No. WO2002017924 to Tew, et al., and as described in Wang, et al., Tetrahedron, 65:32 (2009).

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; Via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### Administration and Formulation

In an embodiment, there is provided method for administering a pharmaceutical composition comprising a pharmaceutically acceptable diluent and a therapeutically effective amount of a compound as described herein, particularly SB-462795, or a pharmaceutically acceptable salt thereof.

The compounds for use in aspects and embodiments of the present invention s described herein can be supplied in the form of a pharmaceutically acceptable salt.

Illustrative pharmaceutically acceptable acid salts of the compounds of the present invention can be prepared from the following acids, including, without limitation formic, acetic, propionic, benzoic, succinic, glycolic, gluconic, glutaric, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. Preferred pharmaceutically acceptable salts include the salts of hydrochloric acid and trifluoroacetic acid.

Illustrative pharmaceutically acceptable inorganic base salts of the compounds of the present invention include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary base salts include aluminum, calcium, lithium, magnesium, potassium, sodium and zinc. Other exemplary base salts include the ammonium, calcium, magnesium, potassium, and sodium salts. Still other exemplary base salts include, for example, hydroxides, carbonates, hydrides, and alkoxides including NaOH, KOH, Na₂CO₃, K₂CO₃, NaH, and potassium-t-butoxide.

Salts derived from pharmaceutically acceptable organic non-toxic bases include salts of primary, secondary, and tertiary amines, including in part, trimethylamine, diethylamine, N,N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

The above salts described herein can be prepared by those skilled in the art by conventional means from the corresponding compounds described herein, particularly from compound SB-462795. For example, the pharmaceutically acceptable salts as described herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The salt may precipitate from solution and be collected by filtration or may be recovered by evaporation of the solvent. The degree of ionization in the salt may vary from completely ionized to almost non-ionized. Other lists of suitable salts are found in Remington's Pharmaceutical Sciences, 17th ed., Mack Publishing Company, Easton, Pa., 1985, p.1418, the disclosure of which is hereby incorporated by reference only with regards to the lists of suitable salts.

The compounds for use in aspects and embodiments of the invention as described herein may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D20, d₆-acetone, d₆-DMSO.

Compounds for use in aspects and embodiments of the invention as described herein may contain one or more asymmetric carbon atoms and can exist as two or more stereoisomers.

Included within the scope of the claimed compounds present invention are all stereoisomers, geometric isomers and tautomeric forms of the compounds described herein, including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound described herein contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes pharmaceutically acceptable isotopically-labelled forms of the compounds described herein, wherein one or more atoms are replaced by atoms having the same atomic number, but an atomic mass or mass number different from the atomic mass or mass number usually found in nature.

The compounds as described herein, particularly SB-46-2795, or a pharmaceutically acceptable salt thereof, and pharmaceutical formulations thereof as described herein, may be used in combination with or include one or more other therapeutic agents for the treatment or prophylaxis of a viral disease in a subject in need thereof caused by a virus. The one or more other therapeutic agents may be selected from anti-inflammatory agents, anticholinergic agents (particularly an M₁/M₂/M₃ receptor antagonist), β₂-adrenoreceptor agonists, anti-infective agents such as antibiotics, antiviral agents, anti-retroviral agents, or antihistamines, 3C protease inhibitors, P4IKB inhibitors, transmembrane protease, serine 2 (TMPRSS2) inhibitors including camostat and nafamostat, immunomodulatory compounds that may ameliorate and/or prevent a “cytokine storm” including, but not limited to, hydroxychloroquine, chloroquine, and tafenoquine, and PDE4 inhibitors. Embodiments of the invention thus providea combination for the treatment or prophylaxis of a viral disease in a subject caused by a virus, the combination comprising a first therapeutic agent that is a compound described herein, particularly SB-462795, or a pharmaceutically acceptable salt, solvate or physiologically functional derivative thereof, together with a second therapeutic agent, wherein the second therapeutic agent is an agent being investigated or currently being administered for treatment of a coronavirus, particularly SARS-CV-2-2, including chloroquine, hydrochloriquine, Tafenoquine,Kaletra (ritonavir and lopinavir), interferon-α-2b, remdesivir, favipiravir, Actemra (tocilizumab), Kevzara (sarilumab). In particular embodiments the second therapeutic agent is an antiviral agent, particularly an anti-retroviral agent, an antibacterial agent, an antihistamine, an antimalarial agent, an anti-inflammatory agent, an immune modulator a bronchodilator, an anti-asthmatic agent, an RNA polymerase inhibitor, a heme polymerase inhibitor, an HIV protease inhibitor, a cysteine (3C or C3) protease inhibitor including a picornain 3C inhibitor or a 3C-like (3CL) protease inhibitor, a phosphodiesterase-4 (PDE-4) isnhibitor, a phosphatidyl inositol 4-kinase beta (PI4KB) inhibitor, a B-adrenoreceptor agonist, or an anticholinergic agent.

Examples of β2-adrenoreceptor agonists suitable for use in combination with a compound as described herein, particularly SB-462795, include salmeterol (which may be a racemate or a single enantiomer such as the R-enantiomer), salbutamol (which may be a racemate or a single enantiomer such as the R-enantiomer), formoterol (which may be a racemate or a single diastereomer such as the R,R-diastereomer), salmefamol, fenoterol, carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol.

Suitable anti-inflammatory agents for use in combination with a compound as described herein, particularly SB-462785, include anti-inflammatory rheumatoid arthritis immunotherapies, including tocilizumab and sarilumab, corticosteroids. Examples of corticosteroids which may be used in combination with the compounds described herein are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid Σ-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carboxylic acid cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. In one embodiment corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carboxylic acid cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester. In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Examples of suitable corticosteroids for use in combination with a compound as described herein for the treatment or prophylaxis of a viral disease in a subject caused by a virus, may include those described in WO2002/088167, WO2002/100879, WO2002/12265, WO2002/12266, WO2005/005451, WO2005/005452, WO2006/072599 and WO2006/072600.

Non-steroidal compounds having glucocorticoid agonism that may possess selectivity for transrepression over transactivation and that may be useful in combination with a compound as described herein, including SB-462795, for the treatment or prophylaxis of a viral disease in a subject caused by a virus, include those covered in the following published patent applications and patents: WO1998/54159, WO2000/66590, WO2001/16128, WO2002/02565, WO2003/059899, WO2003/061651, WO2003/082280, WO2003/082787, WO2003/082827, WO2003/086294, WO2003/101932, WO2003/104195, WO2004/005229, WO2004/009017, WO2004/018429, WO2004/026248, WO2006/000398, WO2006/000401, WO2006/015870, WO2006/108699, WO2007/000334, WO2007/054294, WO2007/122165, WO2007/144327 and WO2008/000777.

Examples of suitable anti-inflammatory agents for use in combination with a compound as described herein, particularly SB-462795 for the treatment or prophylaxis of a viral disease in a subject caused by a virus, include non-steroidal anti-inflammatory drugs (NSAID's).

Examples of suitable NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example montelukast), iNOS inhibitors, tryptase and elastase inhibitors, beta-2 integrin antagonists and adenosine receptor agonists or antagonists (e.g. adenosine 2a agonists), cytokine antagonists (for example chemokine antagonists, such as a CCR3 antagonist) or inhibitors of cytokine synthesis, or 5-lipoxygenase inhibitors.

In one embodiment, iNOS (inducible nitric oxide synthase) inhibitor for use in combination with a compound as described herein, particularly SB-462795 for the treatment or prophylaxis of a viral disease in a subject caused by a virus, include the iNOS inhibitors disclosed in WO1993/13055, WO1998/30537, WO2002/50021, WO1995/34534 and WO1999/62875.

Examples of CCR3 inhibitors for use in combination with a compound as described herein, particularly SB-462795 for the treatment or prophylaxis of a viral disease in a subject caused by a virus, include those disclosed in WO2002/26722.

Examples of PDE4 inhibitory compounds for use in combination with a compound as described herein, particularly SB-462795 for the treatment or prophylaxis of a viral disease in a subject caused by a virus, include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep. 1996; this patent and the compounds it discloses are incorporated herein in full by reference.

Examples of anticholinergic agents for use in combination with a compound as described herein, particularly SB-462795 for the treatment or prophylaxis of a viral disease in a subject caused by a virus, include those compounds that act as antagonists at the muscarinic receptors, in particular those compounds which are antagonists of the M₁ or M₃ receptors, dual antagonists of the M₁/M₃ or M₂/M_{3,} receptors or pan-antagonists of the M₁/M₂/M₃ receptors. Exemplary anticholinergic agents for use in combination with a compound as described herein, particularly SB-462795 for the treatment or prophylaxis of a viral disease in a subject caused by a virus, include for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO2001/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (for example, as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Additional suitable anti-cholinergic compounds are disclosed in WO2005/037280, WO2005/046586, WO2005/104745, and WO2005/009439, incorporated herein by reference. The present combinations include, but are not limited to: (3-endo)-3-(2,2-di-2-thienylethenyl)-8,8-dimethyl-8-azoniabicyclo[3.2.1]octane iodide; (3-endo)-3-(2-cyano-2,2-diphenylethyl)-8,8-dimethyl-8-azoniabicyclo[3.2.1]octane bromide; 4-[hydroxy(diphenyl)methyl]-1-{2-[(phenylmethyl)oxy]ethyl}-1-azoniabicyclo[2.2.2]octane bromide; and (1R,5S)-3-(2-cyano-2,2-diphenylethyl)-8-methyl-8-{2-[(phenylmethyl)oxy]ethyl}-8-azoniabicyclo[3.2.1]octane bromide.

Examples of antiviral agents suitable for combination with compounds described herein, particularly SB-462795 or a pharmaceutically acceptable salt thereof, for the treatment or prophylaxis of a viral disease in a subject caused by a virus, include but are not limited to capsid inhibitors, ribavirin, palivizumab, GS-5806, AL-8176, MEDI-8897, REGN-2222, ALX-0171, ALN-RSV01, AK0529, Favipiravir (T-705), VX-878 (3N3-872), S-033188, FI6, CR9114, MHAA4549A (39.29), VIS410, lamivudine, interferons, nucleoside analogues, and acyclic nucleoside phosphonate analogues, NS3/4A protease inhibitors, NS5A inhibitors, NS5B polymerase inhibitors, 5-substituted 2′-deoxyuridine analogues, entry inhibitors, nucleoside analogues, pyrophosphate analogues, acyclic guanosine analogues, matrix 2 protein inhibitors, RNA polymerase inhibitors, and neuraminidase inhibitors, acyclic guanosine analogues, acyclic nucleoside phosphonate analogues, pyrophosphate analogues, oligonucleotides, nucleoside analogues, 5-substituted 2′-deoxyuridine analogues, antibodies, imiquimod, sinecatechins, podofilox, acyclovir, TMPRSS2 inhibitors, PI4KB inhibitors, 3C protease and 3CL-protease inhibitors, camostat, nafamostat and tafenoquine. Additional antiviral agents suitable for combination with cathepsin inhibitor compounds described herein, particularly SB-462795, for use in the treatment or prophylaxis of a viral disease in a subject caused by a virus, include the antiviral compounds described by E. de Clercq and G. Li in Clin Microbiol Rev (2016), 29:695-747 (“Approved Antiviral Drugs over the Past 50 Years”).

Examples of anti-retroviral agents suitable for combination with compounds described herein, particularly SB-462795 or a pharmaceutically acceptable salt thereof, for the treatment or prophylaxis of a viral disease in a subject caused by a virus, include dolutegravir, cabotegravir, cabotegravir LA, remdesivir, is zidovudine, didanosine, lamivudine, zalcitabine, abacavir, stavudine, adefovir, adefovir dipivoxil, fozivudine, elsufavirine, VM-1500A, ABX464, rilpivirine, todoxil, emtricitabine, alovudine, amdoxovir, elvucitabine, nevirapine, delavirdine, efavirenz, loviride, immunocal, oltipraz, capravirine, lersivirine, doravirine, 3TC, AZT, TMC-278, TMC-125, etravirine, saquinavir, ritonavir, indinavir, nelfinavir, amprenavir, fosamprenavir, fostemsavir, EFdA (MK-8591), MK-8583, MK-8527, MK-8558, GS-9131, GS-9148, GS-PI1, GS-CA1, GS-PS1, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, GSK2248761, GSK3640254, GSK1349572, GSK1265744, GSK3732394 (Combinectin), vicriviroc (Sch-C), Sch-D, TAK779, maraviroc, TAK449, didanosine, tenofovir, lopinavir, darunavir, ibulizamab, PRO-140 (leronlimab), UB-421, VRC01, VRC01/LS, VRC07-523LS, 3BNC117/LS, 10-1074/LS, PGDM1400, PGT121, 10E8.4/iMab,

Examples of antibacterial agents suitable for combination with compounds described herein, particularly SB-462795 or a pharmaceutically acceptable salt thereof, for the treatment or prophylaxis of a viral disease in a subject caused by a virus, include those described by The Pew Charitable Trusts at https://www.pewtrusts.org/en/research-and-analysis/data-visualizations/2014/antibiotics-currently-in-clinical-development. In particular, examples of suitable antibacterial agents for use in combination with compounds as described herein for use or for methods of treatment of viral disease described herein, include but are not limited to eravacycline, omadacycline, iclaprim, S-649266, imipenem, cilastatin, a dehydropeptidase inhibitor, carbapeneum, relabactam, lefamulin (BC-3781), lascufloxacin (KRP-AM1977), CRS3123, delpazolid (LCB01-0371), ETX0282CPDP, SPR741, TP-271, TP-6076, WCK 5222, ACX-362E, afabidin, ARV-1801, brilacidin, CG-549, finafloxacin, MDB-BP-3, gepotidacin (GSK2140944), cephalosporin, β-lactamase inhibitors, tetracyclines, pleuromutilins, fluoroquinolones, a 2,4-aminopyrimidine, a diaryldiamine, an oxazolidone, a polymyxin, a benzofuran naphthyridine, a fusidane, a defensin mimetic, a benzyl pyridinone, a distamycin, a quinolone, a rifamycin-quinolone hybrid, a triazaacenaphthylene, a bis-benzimidazole, an antimicrobial peptide mimetic, and a macrolide.

Examples of 3C protease or 3CL inhibitors suitable for combination with compounds described herein, particularly SB-462795 or a pharmaceutically acceptable salt thereof, for the treatment or prophylaxis of a viral disease in a subject caused by a virus, include the compounds disclosed in WO2018/042343, published Apr. 19, 2018.

Examples of PI4KB inhibitors suitable for combination with compounds described herein, particularly SB-462795 or a pharmaceutically acceptable salt thereof, for the treatment or prophylaxis of a viral disease in a subject caused by a virus, include compounds disclosed in WO2019/141694, published on Jul. 25, 2019.

One embodiment provides a combination comprising a compound described herein, more particularly SB-462705, or a pharmaceutically acceptable salt thereof, for the treatment or prophylaxis of a viral disease in a subject caused by a virus, together with an H1 antagonist. Examples of suitable H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly azelastine, cetirizine, levocetirizine, efletirizine and fexofenadine.

In another embodiment the invention provides a combination comprising a compound described herein, more particularly SB-462705, or a pharmaceutically acceptable salt thereof for the treatment or prophylaxis of a viral disease in a subject caused by a virus, together with an H3 antagonist (and/or inverse agonist). Examples of suitable H3 antagonists include, for example, those compounds disclosed in WO2004/035556, WO2006/045416, WO2006/090142, WO2006/125665, WO2007/009739 and WO2007/009741. In another embodiment the invention provides a combination comprising a compound described herein, or a pharmaceutically acceptable salt thereof for the treatment or prophylaxis of a viral disease in a subject caused by a virus, together with an H1/H3 dual antagonist (and/or inverse agonist). Examples of H1/H3 dual antagonists include, for example, those compounds disclosed in WO2004/035556, WO2007/071691, WO2007/122156 and WO2007/135081.

It will be clear to a person skilled in the art that, where appropriate, the one or more additional therapeutic agent(s) may be used in the form of salts, for example as alkali metal or amine salts or as acid addition salts, or prodrugs, or as esters, for example lower alkyl esters, or as solvates, for example hydrates to optimize the activity and/or stability and/or physical characteristics, such as solubility, of the therapeutic agent. It will be clear also that, where appropriate, the therapeutic agents may be used in optically pure form.

An embodiment of the invention thus provides a combination comprising a compound described herein, more particularly SB-462705, or a pharmaceutically acceptable salt, solvate or physiologically functional derivative thereof, for the treatment or prophylaxis of a viral disease in a subject caused by a virus, together with a PDE4 inhibitor, for the treatment or prophylaxis of a viral disease in a subject caused by a virus.

An embodiment of the invention thus provides a combination comprising a compound described herein or a pharmaceutically acceptable salt, solvate or physiologically functional derivative thereof together with a β₂-adrenoreceptor agonist, for the treatment or prophylaxis of a viral disease in a subject caused by a virus.

An embodiment of the invention thus provides a combination comprising a compound described herein or a pharmaceutically acceptable salt, solvate or physiologically functional derivative thereof together with a corticosteroid, for the treatment or prophylaxis of a viral disease in a subject caused by a virus.

An embodiment of the invention thus provides a combination comprising a compound described herein or a pharmaceutically acceptable salt, solvate or physiologically functional derivative thereof together with a non-steroidal GR agonist, for the treatment or prophylaxis of a viral disease in a subject caused by a virus.

An embodiment of the invention thus provides a combination comprising a compound described herein or a pharmaceutically acceptable salt, solvate or physiologically functional derivative thereof together with an anticholinergic agent, for the treatment or prophylaxis of a viral disease in a subject caused by a virus.

An embodiment of the invention thus provides a combination comprising a compound described herein or a pharmaceutically acceptable salt, solvate or physiologically functional derivative thereof together with an antihistamine, for the treatment or prophylaxis of a viral disease in a subject caused by a virus.

An embodiment of the invention thus provides a combination comprising a compound described herein or a pharmaceutically acceptable salt, solvate or physiologically functional derivative thereof together with a PDE4 inhibitor and a β₂-adrenoreceptor agonist, for the treatment or prophylaxis of a viral disease in a subject caused by a virus.

An embodiment of the One embodiment provides a combination comprising a compound described herein, particularly SB-462795 or a pharmaceutically acceptable salt thereof, and a 3C protease inhibitor, for the treatment or prophylaxis of a viral disease in a subject caused by a virus.

One embodiment provides a combination comprising a compound described herein, particularly SB-462795 or a pharmaceutically acceptable salt thereof, and a PI4KB inhibitor, for the treatment or prophylaxis of a viral disease in a subject caused by a virus.

One embodiment provides a combination comprising a compound described herein, particularly SB-462795 or a pharmaceutically acceptable salt thereof, and a TMPRSS2 inhibitor, for the treatment or prophylaxis of a viral disease in a subject caused by a virus.

One embodiment provides a combination comprising a compound described herein, particularly SB-462795 or a pharmaceutically acceptable salt thereof, and a trypsin protease inhibitor or inhibitor of a trypsin-like protease, for the treatment or prophylaxis of a viral disease in a subject caused by a virus.

One embodiment provides a combination comprising a compound described herein, particularly SB-462795 or a pharmaceutically acceptable salt thereof, and a furin inhibitor or an inhibitor of a related proprotein convertase (PC) protease e.g. peptide inhibitors such as decanoyl-RVKR-chloromethylketone (CMK) or small molecule inhibitors such as naphthofluorescein, for the treatment or prophylaxis of a viral disease in a subject caused by a virus.

The combinations referred to above may conveniently be presented for use in the form of a pharmaceutical formulation and thus pharmaceutical formulations comprising a combination as defined above together with a pharmaceutically acceptable diluent or carrier represent a further aspect of the invention.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The compounds of the present invention may be administered as prodrugs. Thus, certain derivatives of any of the compounds described herein, which may have little or no pharmacological activity themselves can, when administered into or onto the body, be converted into compounds having the desired activity, for example, by hydrolytic cleavage. Such derivatives are referred to as ‘prodrugs’.

Pharmaceutical compositions or formulations include solid, semi-solid, liquid and aerosol dosage forms, such as, e.g., tablets, capsules, powders, liquids, suspensions, aerosols or the like. The chemical entities can also be administered in sustained or controlled release dosage forms, for prolonged and/or timed, pulsed administration at a predetermined rate. In certain embodiments, the compositions are provided in unit dosage forms suitable for single administration of a precise dose.

The compounds described herein, particularly SB-462795 or a pharmaceutically acceptable salt thereof can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). Liquid carriers, for injectable solutions, include water, saline, aqueous dextrose, and glycols.

If desired, the pharmaceutical composition comprising a compound described herein, particularly SB-462795 or a pharmaceutically acceptable salt thereof, can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Saences, Mack Publishing Company, Easton, Pennsylvania.

In certain embodiments, the pharmaceutical compositions comprising a compound described herein, particularly SB-462795 or a pharmaceutically acceptable salt thereof, will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) may be encapsulated in a gelatin capsule.

Liquid pharmaceutically administrable compositions comprising a compound described herein, particularly SB-462795 or a pharmaceutically acceptable salt thereof can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution. Liquid carriers, for injectable solutions, include water, saline, aqueous dextrose, and glycols.

Pharmaceutical compositions comprising a compound described herein, particularly SB-462795 or a pharmaceutically acceptable salt thereof, may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

In general, the compounds described herein, particularly SB-462795 or a pharmaceutically acceptable salt thereof, will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used, the route and form of administration, and other factors. The drug can be administered more than once a day, such as once or twice a day.

Therapeutically effective amounts of the compounds described herein, particularly SB-462795 or a pharmaceutically acceptable salt thereof, may range from approximately 0.01 to 200 mg per kilogram body weight of the recipient per day; such as about 0.01-100 mg/kg/day, for example, from about 0.1 to 50 mg/kg/day. Thus, for administration to a 70 kg person, the dosage range may be about 7-3500 mg per day.

In certain embodiments, a compound as described herein, more particularly SB-462795, or a pharmaceutically acceptable salt thereof, may be administered topically via nasal or oral routes. This includes the application of a compound described herein by inhalation to the lung or nose. Formulations suitable for topical administration for inhalation include liquid or semi-liquid preparations, including formulations suitable for inhalation by penetration through nasal passages and/or to the lung or to the site of inflammation include preparations such as drops suitable. The active ingredient may comprise, for topical administration by inhalation, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

In certain embodiments, drops may comprise sterile aqueous or oily solutions or suspensions and may be prepared by dissolving the active ingredient comprising a compound as described herein, more particularly SB-462795, or a pharmaceutically acceptable salt thereof, in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. In embodiments, the solution for mixing with a compound as described herein, more particularly SB-462795, or a pharmaceutically acceptable salt thereof, may be sterilized by filtration and transferred to the container by an aseptic technique. In embodiments, examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Any of the compounds herein may be administered parenterally, that is by intravenous, intramuscular or subcutaneous, administration. The subcutaneous and intramuscular forms of parenteral administration are generally preferred. Appropriate dosage forms for such administration may be prepared by conventional techniques. Any of the compounds described herein may also be administered by inhalation, which is by intranasal and oral inhalation administration. Appropriate dosage forms for such administration, such as an aerosol formulation or a metered dose inhaler, may be prepared by conventional techniques. In one embodiment of the present invention, any of the compounds of the present invention are delivered via oral inhalation or intranasal administration. Appropriate dosage forms for such administration, such as an aerosol formulation or a metered dose inhaler, may be prepared by conventional techniques.

Embodiments of the invention provide formulations for administration by inhalation of a compound as described herein, more particularly SB-462795, or a pharmaceutically acceptable salt thereof, delivered in the form of an aerosol spray presentation from pressurized packs or a nebulizer, with the use of a suitable propellant, e.g. dichlorodifluoromethane, trichlorofluoromethane, dichlorotetrafluoroethane, a hydrofluoroalkane such as tetrafluoroethane or heptafluoropropane, carbon dioxide or other suitable gas. Certain embodiments provide a formulation for inhalation using a pressurized aerosol, wherein the dosage unit may be determined by providing a valve to deliver a metered amount of a compound as described herein, more particularly SB-462795, or a pharmaceutically acceptable salt thereof. In certain embodiments, capsules and cartridges of e.g. gelatin for use in an inhaler or insufflator may be formulated containing a powder mix of a compound as described herein, more particularly SB-462795, or a pharmaceutically acceptable salt thereof, and a suitable powder base such as lactose or starch.

Certain embodiments of the invention provide a compound as described herein, more particularly SB-462795, or a pharmaceutically acceptable salt thereof, as dry powder compositions for topical delivery to the lung by inhalation. Preferably, the dry powder compositions may, for example, be presented in capsules and cartridges of for example gelatin, or blisters of for example laminated aluminum foil, for use in an inhaler or insufflator. In certain embodiments, powder blend formulations contain a powder mix for inhalation of a compound as described herein, more particularly SB-462795, or a pharmaceutically acceptable salt thereof, and a suitable powder base (carrier/diluent/excipient substance) such as mono-, di or poly-saccharides (e.g. lactose or starch). In certain embodiments, use of lactose in the powder composition is preferred.

In other embodiments, each capsule or cartridge for the powder composition may generally contain between 20 μg-10 mg of any of a compound as described herein, more particularly SB-462795, or a pharmaceutically acceptable salt thereof, optionally in combination with another therapeutically active ingredient. Alternatively, a compound of the invention as described herein, more particularly SB-462795, or a pharmaceutically acceptable salt thereof, for use in a powder composition may be presented without excipients. In embodiments, the packing/medicament dispenser is of a type such as a reservoir dry powder inhaler (RDPI), a multi-dose dry powder inhaler (MDPI), and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to a delivery position.

In embodiments, a metered dose inhaler (MDI) for a medicament dispenser suitable for dispensing medicament comprising a compound as described herein, more particularly SB-462795, or a pharmaceutically acceptable salt thereof, in aerosol form may be used, wherein the medicament is comprised in an aerosol container suitable for containing a propellant-based aerosol medicament formulation. In certain embodiments, the aerosol container may be provided with a metering valve, for example a slide valve, for release of the aerosol form medicament formulation to the patient. In certain embodiments, the aerosol container may be designed to deliver a predetermined dose of medicament upon each actuation by means of the valve, which can be opened either by depressing the valve while the container is held stationary or by depressing the container while the valve is held stationary. In embodiments, the metering volumes are typically from 10 to 100 μL, such as 25 μL, 50 μL or 63 μL.

In certain embodiments, the valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

In certain embodiments, the valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined there between and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085.

By multi-dose dry powder inhaler (MDPI) is meant an inhaler suitable for dispensing medicament comprising a compound as described herein, more particularly SB-462795, or a pharmaceutically acceptable salt thereof, in dry powder form, wherein the medicament is comprised within a multi-dose pack containing (or otherwise carrying) multiple, define doses (or parts thereof) of medicament. In a preferred embodiment, the carrier has a blister pack form, but it could also, for example, comprise a capsule-based pack form or a carrier onto which medicament has been applied by any suitable process including printing, painting and vacuum occlusion.

In embodiments of multi-dose delivery of medicament as described herein, the formulation of a dry powder composition comprising a compound as described herein, more particularly SB-462795, or a pharmaceutically acceptable salt thereof, can be pre-metered or metered in use. An example of a unit-dose device is Rotahaler (see U.S. Pat. Nos. 4,353,656 and 5,724,959, the disclosures of which are hereby incorporated by reference).

Embodiments of the invention provide a compound as described herein, more particularly SB-462795, or a pharmaceutically acceptable salt thereof, formulated as an effective pharmaceutical nasal composition, wherein the medicament is delivered readily to all portions of the nasal cavities (the target tissues) where it performs its pharmacological function. In embodiments, the medicament comprising a compound as described herein, more particularly SB-462795, or a pharmaceutically acceptable salt thereof, remains in contact with the target tissues for relatively long periods of time. Preferably, the medicament remains in contact with the target tissues for extended periods. In embodiments, the medicament comprising a compound as described herein, more particularly SB-462795, or a pharmaceutically acceptable salt thereof, is capable of resisting the forces in the nasal passages that function to remove particles from the nose. Such forces, referred to as ‘mucociliary clearance’, are recognized as being extremely effective in removing particles from the nose in a rapid manner, for example, within 10-30 minutes from the time the particles enter the nose.

Embodiments of the invention provide spray compositions for topical delivery to the lung by inhalation and may, for example, be formulated as aqueous solutions or suspensions or as aerosols delivered from pressurized packs, such as a metered dose inhaler, with the use of a suitable liquefied propellant. Aerosol compositions suitable for inhalation can be either a suspension or a solution and generally contain a compound described herein, preferably SB-462795 or a pharmaceutically acceptable salt thereof, optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants, e.g., oleic acid or lecithin and cosolvents, e.g. ethanol. Pressurized formulations will generally be retained in a canister (e.g. an aluminum canister) closed with a valve (e.g. a metering valve) and fitted into an actuator provided with a mouthpiece.

One embodiment on the invention provides a spray-dried dispersion formulation of SB-462795 or a pharmaceutically acceptable salt thereof, for administration by inhalation. Medicaments for administration by inhalation desirably have a controlled particle size. The optimum particle size for inhalation into the bronchial system is usually 1-10 μm, preferably 2-5 μm. Particles having a size above 20 pm are generally too large when inhaled to reach the small airways. To achieve these particle sizes the particles of the active ingredient as produced may be size reduced by conventional means e.g., by micronization. The desired fraction may be separated out by air classification or sieving. Suitably, the particles will be crystalline in form. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 μm and not less than 15% will have a MMD of less than 15 μm. Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulization may be formulated with an aqueous vehicle with the addition of agents such as acid or alkali, buffer salts, isotonicity adjusting agents or antimicrobials. They may be sterilized by filtration or heating in an autoclave, or presented as a non-sterile product. Suitably, administration by inhalation may preferably target the organ of interest for respiratory diseases, i.e. the lung, and in doing so may reduce the efficacious dose needed to be delivered to the patient. In addition, administration by inhalation may reduce the systemic exposure of the compound thus avoiding effects of the compound outside the lung.

The following examples describe methods and experimental examples that support aspects and embodiments of the invention as described herein.

### BIOLOGICAL EXAMPLES

### Example 1. Biochemical Inhibition of Cathepsins

Inhibition of cathepsin activity was done essentially as described in (B Votta et al., J. Bone Miner. Res. (2015) 16, 478). Enzymes were incubated with peptide substrates containing a fluorescent tag on one end and a quencher tag at the other and catalysis was quantitated by the increase in fluorescence signal. A substrate of Cbz-Phe-Arg-AMC was used in a reaction buffer of 50 mM MES pH 6.5, 5 mM EDTA, 10 mM cysteine in 2% DMSO. Linear portions of the initial velocity curves were analyzed to generate steady state kinetic constants.

TABLE 1
Protease inhibitor activity of relacatib (SB-462795) (Ki,
nM). (see Yamashita J Med Chem, 2006 49, 1597-1612)

| | | | Cat V | | | | | | |
|---|---|---|---|---|---|---|---|---|---|
| Ki(nM) | Cat K | Cat L | (L2) | Cat S | Cat B | Cat G | Cat D | Calpain | Caspase 3-4 |
| Human | 0.04 | 0.07 | 0.05 | 1.6 | 15 | >30,000 | >10,000 | 310 | >10,000 |
| Monkey | 0.04 | 0.3 | 0.7 | ND | 11 | ND | ND | ND | ND |
| Rat | 8.0 | 0.2 | ND | 5.5 | 67 | ND | ND | ND | ND |
| Mouse | 15 | 0.2 | ND | ND | ND | ND | ND | ND | ND |

### Example 2. Viral Inhibition by Relacatib (SB-462795)

### Cells

HeLa (CCL-2), MRC5 (CCL-171), HFF (SCRC-1041), Vero (CCL-81), BHK-21 (CCL-10) cells were purchased from the American Type Culture Collection. HeLa, Vero and HFF cells were cultured in T225 (Corning Life Sciences) flasks in Minimum Essential Medium (MEM, Corning Life Sciences) supplemented with 10% fetal bovine serum (FBS, Hyclone, GE Life Sciences), 1% L-glutamine, 10 mM HEPES (Sigma Aldrich), 1% non-essential amino acids (ThermoFischer Scientific), and 1% penicillin/streptomycin (Corning). HFF cells were also supplemented with Sodium pyruvate solution (Sigma Aldrich). MRC5 and BHK-21 cells were cultured in T225 (Corning) flasks in Minimum Essential Medium (MEM, Corning Cellgro) supplemented with 10% fetal bovine serum (FBS, Hyclone). Sub-confluent culture was passaged and split every 3 days, not exceeding 16-19 passages. Cells were detached using 0.25% Trypsin/0.5 mM EDTA solution (Sigma Aldrich) and seeded in assay plates for dose response studies 24 hours before treatment (see Table 2 for seeding densities). Whole blood was purchased from the New York Blood Center and human PBMCs were isolated using Ficoll Paque Plus (GE Healthcare). When indicated, CD14 positive monocytes were further isolated using human CD14 microbeads (Miltenyi Biotec). To differentiate cells into macrophages, PBMCs or CD14+monocytes were seeded in 10 cm plates in RPMI 1640 medium (FisherScientific) supplemented with 10% heat inactivated FBS (ThermoFisher Scientific), and 1% penicillin-streptomycin (Corning), and after 3 days treated with recombinant human granulocyte monocyte-colony stimulating factor (GM-CSF,10 ng/ml). Two days later, cells were supplemented with fresh media and GM-CSF. Six or seven days after seeding, cells were detached, counted and seeded in 96-well plates (Greiner Bio-One) for doe response studies.

### Cell-Based Infection Assays:

Experiments with infectious viruses were performed in USAMRIID under appropriate biocontainment conditions. Virus stocks were all prepared and characterized at USAMRIID.

Cells were seeded at the indicated density (Table 2) in 384-well imaging plates (Aurora) or 96-well plates (Greiner Bio-One) at 20-24 hours prior to treatment using the automated Multidrop Combi dispenser (ThermoFisher Scientific). SB-462795 was tested at 8-10 doses in at least two replicates starting at 10-100 μM with a 2- or 3-fold step dilution (see Table 2 for starting concentration and fold dilution of each study). Each dose was added directly to the assay wells from a 10 mM stock solution in 100% DMSO using the HP D300 digital dispenser (Hewlett Packard). The final DMSO concentration in each well was normalized to 1%. On each plate, sixteen wells were not infected with virus and served as a ‘no virus’ control for normalization (0% virus infection). Additional sixteen wells were infected with virus but treated only with 1% DMSO and served as a high infection control for normalization (100% virus infection). Two hours after treatment assay plates were transferred to the biosafety level (BSL)-3 or 4 suite and inoculated with the indicated virus at the indicated multiplicity of infection (MOI). The MOI was calculated based on the average doubling time of cells (16 hours) and was selected to achieve 60-80% infection rate at the assay endpoint. Following virus inoculation, assay plates were incubated at 37° C. with 5% CO₂ for 20 (VEEV and CHIKV), 24 (NiV) or 48 hours (EBOV, MARV, LASV, SARS-CoV, MERS-CoV, WNV, HTNV). Cells were then fixed in 10% buffered formalin for at least 48 hours before immunostaining.

TABLE 2
Broad spectrum antiviral activity of relacatib (SB-462795).

| Family | Virus | Cell type | EC50 [μM] | [symbol] | EC90 [μM] | CC50 [μM] | SI |
|---|---|---|---|---|---|---|---|
| F [symbol] idae | EBOV ( [symbol] wit) | HeLa | 0. [symbol] | 0.0 [symbol] | 1. [symbol] 1 | >2 [symbol] | >234 |
| | | HFF | 0.0 [symbol] | 0.00 [symbol] | 0.304 | >2 [symbol] | > [symbol] 1 |
| | | V [symbol] | 2.89 | 0. [symbol] 10 | 16 [symbol] | >2 [symbol] | > [symbol] |
| | | BHK [symbol] | 0.8 | 0.0 [symbol] | 2 [symbol] | >20 | >2 [symbol] |
| | EBOV | HeLa | 0.360 | 0.0 [symbol] | 7 [symbol] | >10 | >27 [symbol] |
| | (Makona) | HFF | 0.0 [symbol] | 0.010 | 0. [symbol] | >10 | > [symbol] |
| | | M [symbol] | 0.0 [symbol] | 0.0 [symbol] | 0. [symbol] | >10 | > [symbol] |
| | | PBMC | 0. [symbol] | 1.810 | 30 [symbol] | >25 | > [symbol] |
| | | V [symbol] | 1 [symbol] | 0.4 [symbol] | 9.710 | >10 | > [symbol] |
| | EB [symbol] V (mouse- | BH [symbol] | 7.7 | 1 [symbol] | 146 [symbol] | >20 | >2 [symbol] |
| | adapted) | | | | | | |
| | MA [symbol] V | H [symbol] | 0.250 | 0.0 [symbol] | 1 [symbol] | >10 | > [symbol] |
| | | HFF | 0.094 | 0.0 [symbol] | 1. [symbol] | >25 | >266 |
| | | M [symbol] | 0.162 | 0.0 [symbol] | 2.4 [symbol] | >10 | >61. [symbol] |
| | | [symbol] | 0. [symbol] | 0. [symbol] | 3.778 | >30 | > [symbol] |
| | | V [symbol] | [symbol] | 1.8 | 16. [symbol] | >10 | >2.2 |
| Coronaviridae | MERS-CoV | M [symbol] C5 | 0.040 | 0.010 | 0.4 [symbol] | >100 | >2502 |
| | SARS-CoV | V [symbol] | 0. [symbol] | 0.1 [symbol] | 1. [symbol] | >50 | > [symbol] |
| P [symbol] idae | [symbol] V | H [symbol] | 0.015 | 0. [symbol] | 0.0 [symbol] | >10 | > [symbol] |
| Aren [symbol] viridae | [symbol] V | H [symbol] | >100 | — | — | >100 | — |
| H [symbol] viridae | [symbol] V | V [symbol] | >100 | — | — | >100 | — |
| F [symbol] viridae | [symbol] V | H [symbol] | >100 | — | — | >100 | — |
| T [symbol] viridae | [symbol] V | H [symbol] | 42.3 | [symbol] | 1 [symbol] | >100 | >2.4 |
| | [symbol] V | U [symbol] | 8 [symbol] | 4. [symbol] | 1 [symbol] | >100 | > [symbol] .2 |

EC₅₀, 50% effective concentration (μM).
SD, standard deviation,
EC₉₀, 90% effective concentration
CC₅₀: cytotoxic concentration (μM).
SI, selectivity Index. Definitions for virus abbreviations are provided in Methods. [symbol]
indicates data missing or illegible when filed

### High-Content Imaging Assay

Detection and quantification of viral infection in cultured cells was performed using a high-content imaging (HCI) assay by measuring viral antigen production after immunofluorescent labelling. Assay wells were stained for 1 hour with a murine or rabbit monoclonal antibody (Table 3) diluted 1,000-fold in blocking buffer (1×PBS with 3% BSA). Following incubation, the primary antibody was removed and the cells were washed 3 times with 1×PBS. Cells were subsequently incubated for 1 hour with DyLight-488-conjugated goat anti-mouse/rabbit IgG (ThermoFisher Scientific) diluted 1,000-fold in blocking buffer. Nuclei and cytoplasm were stained with Draq5 (HeLa and U2OS cells; ThermoFisher Scientific) or with Hoechst and CellMask (ThermoFisher Scientific), respectively, diluted in 1×PBS. Cell images were acquired using a Perkin Elmer Opera confocal plate reader using a 10X air objective to collect 5 to 15 images per well. Acquired images were analyzed using Acapella (Harmony) PE software. Cells that exhibited antigen signal higher than the selected threshold were counted as positive for viral infection. The ratio of virus-positive cells to the total number of analyzed cells was used to determine the percentage of infection for each well in the assay plates. The effect of compounds on viral infection was assessed using the Genedata software and was calculated as percent inhibition of infection based on the 16 non-infected—(0% infection) and 16 DMSO-treated—(100% infection) control wells on each plate. The resultant % inhibition of infection was calculated for each well and used for the dose—response curve analysis. Multi-parameter regression analysis utilized the Levenberg—Marquardt algorithm for selecting the best model and data point exclusion. R² value quantified fit quality and the fitting strategy was considered acceptable at R²>0.8.

TABLE 3
Primary antibodies used in dose response studies

| Virus | Antibody |
|---|---|
| WNV | mouse monoclonal mm D1-4G2 anti-E |
| VEEV | mouse monoclonal mm 1A4A anti-E2 |
| CHIKV | mouse monoclonal mm 2D21-1 anti-E2 |
| EBOV | mouse monoclonal mm 6D8 anti-GP |
| MARV | mouse monoclonal mm 9G4 anti -GP |
| NiV | rabbit polyclonal anti-G or -F |
| LASV | mouse monoclonal mm L52-161-6 anti-GP |
| MERS-CoV | rabbit RP02 anti-S2 |
| SARS-CoV | rabbit polyclonal anti-S1 (SinBiological Inc) |
| HTNV | mouse monoclonal anti-G2/Gc (bei resources) |

Inhibition of SARS-CoV-2

Antiviral Evaluations—in vitro Cell Based Assay of SARS-CoV1, SARS-CoV2, and MERS in Vero E6 Cells

Applicant separately evaluated SB-462795 (relacatib) and camostat for inhibitory effect against SARS-CoV-1, SARS-CoV-2 and MERS by CPE assay in Vero E6 cells as described below.

### Materials and Methods for Coronavirus Assays

SB-462795 and camostat were supplied as solids and diluted to either 50 mM or 10 mM stock solutions in DMSO and stored at 4° C. until the day of the assays. A set of positive control antiviral compounds were included in each of the assays performed.

### Virus Strains and Cell Lines

The viruses and cell lines utilized for these evaluations were obtained from WRCEVA and American Type Culture Collection (ATCC) (followed by sorting and sub-cloning cells for high expression of ACE2) as listed in Table 4 and Table 5, respectively. The evaluation was performed using a CPE reduction assay to measure antiviral effect and a cell viability assay to measure cytotoxic effect of compounds (non-GLP assays). The day of each assay, a pre-titered aliquot of virus was removed from the freezer (−80° C.) and allowed to thaw to room temperature in a biological safety cabinet. The virus was re-suspended and diluted into tissue culture medium. Cells were sub-cultured twice a week at a split ratio of 1:2 to 1:5 using standard cell culture techniques and the cell culture media as specified below in Table 5. Total cell number and percent viability determinations were performed using a Luna cell viability analyzer and trypan blue dye exclusion. Cell viability was greater than 95% for the cells to be utilized in the assays (see Table 5 for the number of cells seeded per well for each assay).

TABLE 4
Virus Strain used for Antiviral Evaluations

| | | | | | | Assay | |
|---|---|---|---|---|---|---|---|
| | | Cell line | | Positive | | duration | |
| Virus | Strain | Tested | MOI | Control | BSL | (Days) | Endpoint |
| SARS- | Toronto 2 | VeroE6 | 0.002 | Calpain | 3 | 3 | CPE |
| CoV-1 | | | | Inhibitor IV, | | | |
| | | | | Chloroquine, | | | |
| | | | | Remdesivir, | | | |
| | | | | proprietary | | | |
| | | | | compounds | | | |
| SARS- | USA_WA1/ | VeroE6 | 0.002 | Calpain | 3 | 3 | CPE |
| CoV-2 | 2020 | | | Inhibitor IV, | | | |
| | | | | Chloroquine, | | | |
| | | | | Remdesivir, | | | |
| | | | | proprietary | | | |
| | | | | compounds | | | |
| MERS | EMC/2012 | VeroE6 | 0.002 | Calpain | 3 | 3 | CPE |
| | | | | Inhibitor IV, | | | |
| | | | | Chloroquine, | | | |
| | | | | Remdesivir, | | | |
| | | | | proprietary | | | |
| | | | | compounds | | | |

BSL- Biosafety Level

TABLE 5
Cells and Media used for Antiviral Evaluations

| Cell Line | Cat# | Source | Cell No./Well | Cell Source and Assay Medium |
|---|---|---|---|---|
| Vero E6¹ | CRL-1586 | American Type | 4,000 cells per | Culture Medium: MEM² 10% |
| | | Culture | well in 384-well | Heat inactivated FBS³ |
| | | Collection | format | Assay Media: MEM2 2% heat |
| | | (ATCC) | | inactivated FBS³, 1% Pen Strep |

¹Sub-cloned for high expression of ACE2
²DMEM = Dulbecco's Modified Eagle's Medium
³FBS—Fetal Bovine Serum

Materials:
1. Corning 3764 BC black-walled, clear bottom and Greiner 784076 black-walled, low volume 384-plates
2. Promega CellTiter Glo (CTG) (G7573, Promega)
3. Media:
   a. MEM Gibco (#11095)
   b. HI FBS Gibco (#14000)
   c. Pen/Strep Gibco (#15140); 10 U/ml penicillin and 10 ug/ml streptomycin
4. PBS −/− (w/o Ca²⁺ or Mg²⁺)
5. Trypsin-EDTA Gibco (#25300-054)
6. Cells-Vero E6 cells selected for high ACE2 expression.
7. Positive controls:
   a. Calpain Inhibitor IV (Calbiochem #208724)
   b. Chloroquine (Applicant Repository)
   c. Remdesivir (Selleck)

Equipment:
1. Beckman FX
2. Echo and Thermo Combi Liquid handler
3. Matrix WellMate
4. Thermo Fisher Steri-Cult Incubator
5. Microplate Readers-Envision, Spectromax M2, M5 or Gemini or PheraSTAR

### Compound Preparation:

Compound stock solutions (50 μL at 10 or 50 mM in 100% DMSO) were transferred into wells of an empty ECHO plate (stock plate). Compounds were diluted 3-fold by transferring 17 μL of each sample from the stock plate into an adjacent well containing 34 μL DMSO in each well and mixing. This process was repeated to create 8 more wells of serially diluted sample, each well containing a 3-fold diluted sample of the previous well. A 30 nL aliquot for each sample was dispensed into corresponding wells of assay ready plates using an ECH0555 acoustic liquid handling system. The final assay concentration range was as follows: for 10 mM stocks, 10-0.0005 μM; for 50 mM stocks, 50-0.003 μM. DMSO was added to control wells to maintain a consistent assay concentration of 0.1% in all wells.

### Method for Measuring Antiviral Effect of Compounds:

Vero E6 cells were grown in MEM supplemented with 10% HI FBS and harvested in MEM/1% PS supplemented 2% HI FBS on the day of assay. Assay ready plates pre-drugged with test compounds were prepared in the BSL-2 lab by adding 5 μL assay media to each well. The plates and cells were then passed into the BSL3 facility. Cells were batch inoculated with appropriate coronavirus (SARS CoV-1, SARS CoV-2 or MERS) at M.O.I. ˜0.002 which resulted in 5% cell viability 72 (for SARS) or 96 (for MERS) hours post infection. A 25 μL aliquot of virus inoculated cells (4,000 Vero E6 cells/well) was added to each well in columns 3-24 of the assay plates. The wells in columns 23-24 contained only virus infected cells for the 0% CPE reduction controls. Prior to virus inoculation, a 25 μL aliquot of cells was added to columns 1-2 of each plate for the cell only 100% CPE reduction controls. After incubating plates at 37° C./5% CO₂ and 90% humidity for 72 hours, 30 μL of Cell Titer-Glo (Promega) was added to each well. Luminescence was read using a Perkin Elmer Envision plate reader following incubation at room temperature for 10 minutes to measure cell viability. Plates were sealed with a clear cover and surface decontaminated prior to luminescence reading.

### Method for Measuring Cytotoxic Effect of Compounds:

Compound cytotoxicity was assessed in a BSL-2 counter screen as follows: VeroE6 cells in media were added in 25 μl aliquots (4,000 cells/well) to each well of assay ready plates prepared with test compounds as above. Cells only (100% viability) and cells treated with hyamine at 100 μM final concentration (0% viability) serve as the high and low signal controls, respectively, for cytotoxic effect in the assay. DMSO was maintained at a constant concentration for all wells as dictated by the dilution factor of stock test compound concentrations. After incubating plates at 37° C./5% CO2 and 90% humidity for 72 hours, 30 μl Cell Titer-Glo (Promega) was added to each well. Luminescence was read using a BMG PHERAstar plate reader following incubation at room temperature for 10 minutes to measure cell viability.

RESULTS—in vitro Cell Based Assay of SARS-CoV1, SARS-CoV2, and MERS in Vero E6 Cells

Summary data for the reference compounds and test articles is provided in Table 6-8 and 9-12. Table 13 shows the comprehensive data from all four assays.

TABLE 6
Antiviral activity of Reference Compounds in the SARS-CoV-1 CPE assay

| | | | | | | Selectivity |
|---|---|---|---|---|---|---|
| | | | Supplier | Antiviral | Cytotoxicty | Index |
| Compound ID | Batch | Supplier | ID | IC₅₀ (μM) | CC₅₀ (μM) | CC₅₀/IC₅₀ |
| CalpainInh-IV | 01 | Sigma | Calpain | 0.98 | >7.17 | >7.3 |
| | | | Inh-IV | | | |
| Chloroquine | 01 | Inhouse | Chloroquine | 2.02 | >30.00 | >14.9 |
| | | Stock | | | | |
| Remdesivir | 01 | Selleck | Remdesivir | 7.42 | >30.00 | >4.0 |

TABLE 7
Antiviral activity of Reference Compounds in the SARS-CoV-2 CPE assay

| | | | | | | Selectivity |
|---|---|---|---|---|---|---|
| | | | Supplier | Antiviral | Cytotoxicty | Index |
| Compound ID | Batch | Supplier | ID | IC₅₀ (μM) | CC₅₀ (μM) | CC₅₀/IC₅₀ |
| CalpainInh-IV | 01 | Sigma | Calpain | 0.47 | >7.17 | >15.3 |
| | | | Inh-IV | | | |
| Chloroquine | 01 | Inhouse | Chloroquine | 3.71 | >30.00 | >8.1 |
| | | stock | | | | |
| Remdesivir | 01 | Selleck | Remdesivir | 4.15 | >30.00 | >7.2 |

TABLE 8
Antiviral activity of Reference Compounds
in the MERS CPE assay (Standard Incubation)

| | | | | | | Selectivity |
|---|---|---|---|---|---|---|
| | | | Supplier | Antiviral | Cytotoxicty | Index |
| Compound ID | Batch | Supplier | ID | IC₅₀ (μM) | CC₅₀ (μM) | CC₅₀/IC₅₀ |
| CalpainInh-IV | 01 | Sigma | Calpain | 1.07 | >7.17 | >6.7 |
| | | | Inh-IV | | | |
| Chloroquine | 01 | Inhouse | Chloroquine | 20.96 | >30.00 | >1.4 |
| | | stock | | | | |
| Remdesivir | 01 | Selleck | Remdesivir | 12.53 | >30.00 | >2.4 |

TABLE 9
Antiviral activity of Test Compounds
in the SARS-CoV-1 CPE assay

| | | | | Selectivity |
|---|---|---|---|---|
| | | Antiviral | Cytotoxicty | Index |
| Compound ID | Batch | IC₅₀ (μM) | CC₅₀ (μM) | CC₅₀/IC₅₀ |
| relacatib | 01 | 0.23 | >50.00 | >217.4 |
| camostat | 01 | >50.00 | >50.00 | >1 |

TABLE 10
Antiviral activity of Test Compounds
in the SARS-CoV-2 CPE assay

| | | | | Selectivity |
|---|---|---|---|---|
| | | Antiviral | Cytotoxicty | Index |
| Compound ID | Batch | IC₅₀ (μM) | CC₅₀ (μM) | CC₅₀/IC₅₀ |
| relacatib | 01 | 0.02 | >50.00 | >2,500 |
| camostat | 01 | >50.00 | >50.00 | >1 |

TABLE 11
Antiviral activity of Test Compounds in
the MERS CPE assay (Standard Incubation)

| | | | | Selectivity |
|---|---|---|---|---|
| | | Antiviral | Cytotoxicty | Index |
| Compound ID | Batch | IC₅₀ (μM) | CC₅₀ (μM) | CC₅₀/IC₅₀ |
| relacatib | 01 | 0.6 | >50.00 | >83.3 |
| camostat | 01 | >50.00 | >50.00 | >1 |

TABLE 12
Comparison of Antiviral activity of Test Compounds in
the MERS CPE assay (Standard vs preincubation method*)

| | | pre-incubation | std-incubation |
|---|---|---|---|
| Compound ID | Batch | IC₅₀ (μM) | IC₅₀ (μM) |
| relacatib | 01 | 0.68 | 0.6 |
| camostat | 01 | >50.00 | >50.00 |

*Preincubation Method modification: Uninfected cells in 25 μL media were added to predrugged assay ready plates and incubated for four hours before adding 5 μL MERS virus diluted in media.

TABLE 13
Comprehensive Overview of Antiviral activity of Test Compounds in the SARS-CoV-1, SARS-CoV-2, MERS

| | SARS-CoV-1 | | | SARS-CoV-2 | | | MERS | | |
|---|---|---|---|---|---|---|---|---|---|
| | Antiviral | | Selectivity | Antiviral | | Selectivity | Antiviral | | Selectivity |
| Compound | IC₅₀ | Cytotoxicty | Index | IC₅₀ | Cytotoxicty | Index | IC₅₀ | Cytotoxicty | Index |
| ID | (μM) | CC₅₀ (μM) | CC₅₀/IC₅₀ | (μM) | CC₅₀ (μM) | CC₅₀/IC₅₀ | (μM) | CC₅₀ (μM) | CC₅₀/IC₅₀ |
| relacatib | 0.23 | >50.00 | >217.4 | 0.02 | >50.00 | >2,500 | 0.60 | >50.00 | >83.3 |
| camostat | >50.00 | >50.00 | >1 | >50.00 | >50.00 | >1 | >50.00 | >50.00 | >1 |

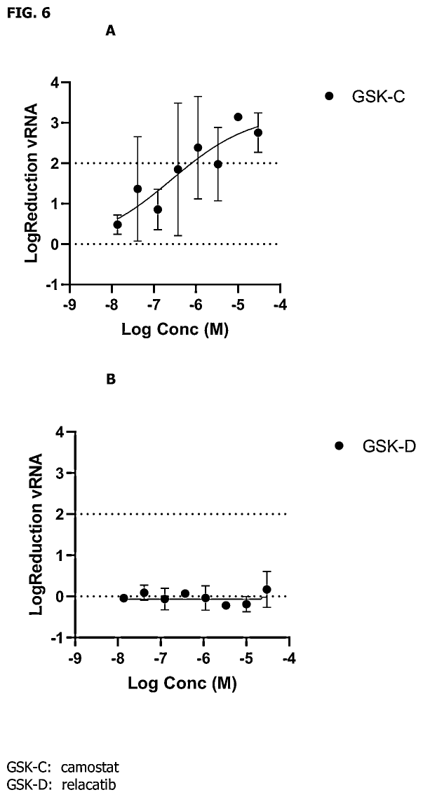

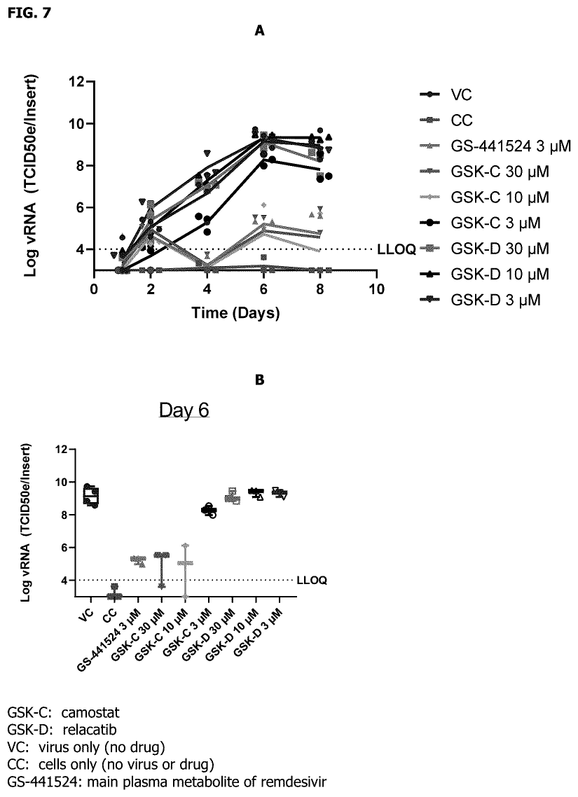

As can be seen in Table 6-12 and summarized in Table 13, and as shown in FIG. 6A (relacatib) and FIG. 6B (camostat). Relacatib showed antiviral activity in Vero E6 cells infected with SARS-CoV-1, SARS-CoV-2 and MERS at IC₅₀ values of 0.23, 0.02 and 0.60 μM, respectively. In contrast, camostat showed no antiviral activity in any of SARS-CoV-1, SARS-CoV-2 and MERS-infected Vero E6 cells. This is consistent with the fact that Vero E6 cells do not express TMPRSS2, allowing relacatib, a cathepsin inhibitor, to target the cathepsin-dependent spike-protein viral entry pathway. In contrast, camostat is expected to target the TMPRSS2 entry pathway of such viruses, but Vero E6 cells do not express TMPRSS2, so observation of no inhibition of SARS-CoV-1, SARS-CoV-2 and MERS by camostat in Vero E6 cells is consistent with this understanding of their mechanisms of action.

Antiviral Evaluations: In vitro Inhibition of SARS-CoV-2 in Calu-3Cells

Antiviral Evaluations—In vitro Inhibition of SARS-CoV-2 in Calu3 Cells (Operetta High Content Imaging System)

Applicant separately evaluated SB-462795, compared to camostat and remdesivir, for inhibitory effect against SARS-CoV-2 in a cellular assay using Calu3 immortalized human lung cells as described below:

### Protocol for SARS-CoV-2 Coronavirus Cellular Assay

SB-462795 (relacatib) and camostat were tested via cellular assays utilizing expression of coronavirus N protein and cell nuclei as end points for imaging (indicators of efficacy and toxicity respectively). Calu3 cells (ATCC, HTB-55) were infected with SARS-CoV-2 coronavirus (βCoV/KOR/KCDC03/2020). Although remdesivir and camostat reduced the expression of N protein, shown below, as measured immunologically (see Table 14) relacatib had no measurable SARS-CoV-2 activity in Calu-3 cells (compare camostat activity in FIG. 6A to relacatib activity in FIG. 6B). The absence of anti-viral activity by SB-462795 against SARS-CoV-2 in Calu-3 cells indicates the virus used alternative entry mechanisms that would not be affected by the cathepsin inhibitor relacatib, presumably by utilizing the TMPRSS2-dependent processing of the spike protein at the cell surface of SARS-CoV-2, and not the cathepsin-dependent spike protein processing in the endosomes of SARS-CoV-2. In preparation for the assay, ten-point, three-fold dose-response curves (DRCs) were generated for the test compound in DMSO with compound concentrations ranging from 0.0025 to 50 μM.

Calu-3 cells were seeded at 2.0×10⁴ cells per well in Eagle's Minimum Essential Medium (EMEM, ATCC) supplemented with 20% heat-inactivated fetal bovine serum (FBS), 1% MEM-Non-Essential Amino Acid solution (Gibco) and 1× Antibiotic-Antimycotic solution (Gibco) in black, 384-well, μClear plates (Greiner Bio-One) 24 hours before the experiment. The cells were maintained at 37° C. with 5% CO₂.

The cells were treated with relacatib, camostat or remdesivir at concentrations ranging from 0.0025 to 50 μM for 1 to 48 hours prior to infection with SARS-CoV-2 at an MOI of 0.03. DMSO was normalized in reaction wells to a final concentration of 0.5%. The plates were incubated at 37° C. for 24 hours before fixing with 4% paraformaldehyde (PFA), 0.25% tritonX-100 solution.

Anti-SARS-CoV-2 Nucleocapsid (N) primary antibody, 488-conjugated goat anti-rabbit IgG secondary antibody and Hoechst 33342 was added prior to immunofluorescence. Images were acquired with an Operetta high-throughput imaging device (Perkin Elmer) and analysed using the Columbus software (Perkin Elmer) to quantify cell numbers and infection ratios. For Operetta endpoint, antiviral activity was normalized to infection control (0.5% DMSO) in each assay plate. Cell viability was measured by counting nucleus in each well and normalizing it to the infection control. ICso values were calculated using nonlinear regression analysis—log[inhibitor] vs. response—Variable slope (four parameters). All IC₅₀ values were measured in triplicate.

### RESULTS—Calu-3

TABLE 14

| Compound | | Exp. 1 | Exp. 2 |
|---|---|---|---|
| ID | Target | IC₅₀ (μM) | IC₅₀ (μM) |
| Remdesivir | | 1.94 | 2.15 |
| GSK-C | Serine | 0.16 | 0.29 |
| | protease | | |
| | inhibitor | | |
| GSK-D | Pan cathepsin | >50 | >50 |
| | inhibitor | | |

GSK-C: camostat
GSK-D: relacatib

As can be seen in Table 14, relacatib has no activity against SARS-CoV-2 in Calu-3 cells, which express TRPMSS2, but ICso values of 0.16 μM and 0.29 μM for Experiment 1 and Experiment 2, respectively, were measured for camostat in Calu-3 cells. See also FIG. 6A (camostat) and FIG. 6B (relacatib).

Antiviral Evaluations: In vitro Inhibition of SARS-CoV-2 in ALI Model

Applicant separately evaluated SB-462795, with and without camostat, for inhibitory effect against SARS-CoV-2 by CPE assay in air-liquid interface (ALI) assays using human lung cells as described below:

Primary human lung cells were used to evaluate test compounds in an air-liquid interface assay essentially as describe by Randell, S. H and Fulcher, M. L. (eds) in Chapter Eight of Epithelial Cell Culture Protocols: Second Edition, Methods in Molecular Biology, vo. 945, DOI 10.1007/978-1-62703-125-7_8 © Springer Science+Business Media, LLC 2012. In short, HBE cells are isolated from the trachea regions as well as the upper bronchus. If possible, branches are isolated that are approximately 2 mm. All cells are all pooled together during these isolations. Resulting cells are grown on a porous support at an air-liquid interface undergo mucociliary differentiation, which reproduces both in vivo morphology and key physiologic processes in the cells. The ALI assay was used as another means to evaluate the antiviral activity of the test compounds described herein against SARS-CoV-2, using essentially the protocol for evaluating SARS-CoV infection as described by Sims, A. C. et al. in J. Virol (December 2005), vol. 79 (24), p. 15511-15524 (“Severe Acute Respiratory Syndrome Coronavirus Infection of Human Ciliated Airway Epithelia: Role of Ciliated Cells in Viral Spread in the Conducting Airways of the Lungs”), 0022-538X/05/$08.00+0 doi:10.1128/JVI.79.24.15511-15524.2005. Primary lung cells (Marsico Lung Institute) were cultured essentially as described by Randell and Fulcher, and infected with SARS-CoV-2 essentially as described by Sims et al., with a MOI of 0.5. SB-462795, camostat, or SB-462795 and camostat were dissolved in dimethylsulfoxide (DMSO), at doses of 3 μM, 10 μM or 30 μM (SB-462795 alone or camostat alone); or 4 μM camostat+30 μM SB-462795t) were administered for 48 hours, and antiviral activity was evaluated by plaque assay compared to controls with 10 μM remdesivir, virus in DMSO solvent and DMSO solvent alone controls.

### RESULTS—ALI Model

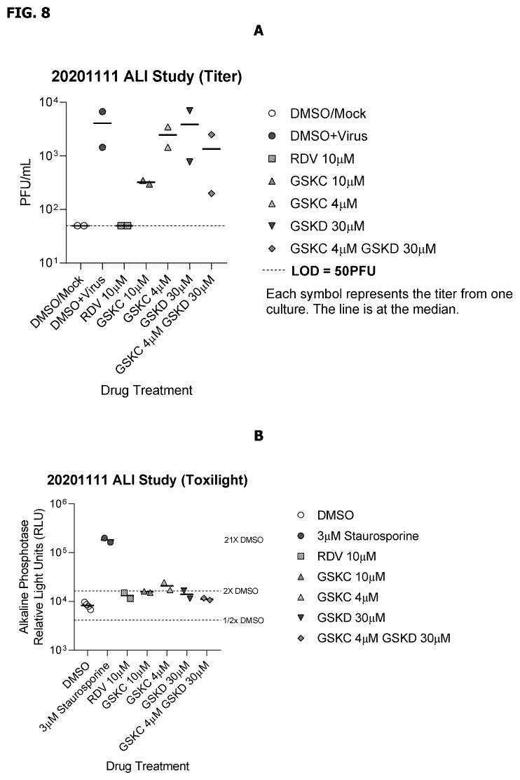

Results are shown in FIG. 8A. As can be seen, Camostat (GSKC in FIG. 8A) exhibited dose-dependent SARS-CoV-2 antiviral activity at 4 μM (lighter upright triangles) and 10 μM (darker upright triangles) compared to 10 μM remdesivir (RDV in FIG. 8A), whereas relacatib (GSKD in FIG. 8A) exhibited essentially no activity at 30 μM (upside down triangles) or 4 μM (squares). However, the combination of relacatib (30 μM) and camostat (4 μM) (diamonds) showed increased activity against SARS-CoV-2 in the ALI study, compared with relacatib alone or camostat alone (see FIG. 8A). Each symbol represents the titer from one culture.

FIG.8B shows cytoxicity as measured in the ALI study, for relacatib (upside down triangles), camostat (upright triangles) and the combination of relacatib with camostat (diamonds), compared to remdesivir main plasma metabolite (squares) and staurosporine, a positive control (circles).

SEQUENCE LISTING

SEQ ID NO: 1: N1 forward primer
5′ GACCCCAAAATCAGCGAAAT 3′
SEQ ID NO: 2: N1 reverse primer
5′ TCTGGTTACTGCCAGTTGAATCTG 3′
SEQ ID NO: 3: N1 Probe Sequence
5′ FAM-ACCCCGCATTACGTTTGGTGGACC-BHQ-1 3′
SEQ ID NO: 4: N2 forward primer
5′ TTACAAACATTGGCCGCAAA 3′
SEQ ID NO: 5: N2 reverse primer
5′ GCGCGACATTCCGAAGAA 3′
SEQ ID NO: 6 N2 Probe Sequence
5′ FAM-ACAATTTGCCCCCAGCGCTTCAG-BHQ-1 3′

1. A method for the treatment or prophylaxis of a viral disease in a subject in need thereof, the method comprising administering to the subject a therapeutically effective amount of a compound of the structure: 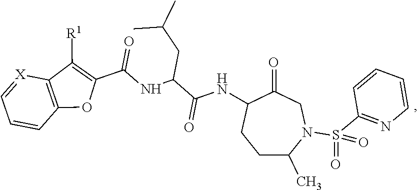
or a pharmaceutically acceptable salt thereof,
wherein:
X is selected from N or CR²; and
R¹ and R² is each independently selected from —H or —(C₁-C₈)alkyl.

2. The method according to claim 1, wherein R¹ is —(C₁-C₈)alkyl.

3. The method according to claim 1, wherein R² is H.

4. The method according to claim 1, wherein R¹ is —CH₃ and R² is H.

5. The method according to claim 1, wherein the compound is: 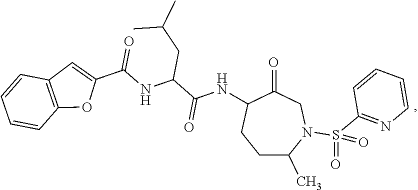 [SMILES: CC(C)CC(NC(=O)c1cc2ccccc2o1)C(=O)NC1CCC(C)N(S(=O)(=O)c2ccccn2)CC1=O]
or a pharmaceutically acceptable salt thereof.

6. The method for the treatment or prophylaxis of a viral disease in a subject in need thereof according to claim 1, the method comprising administering to the subject a therapeutically effective amount of a compound of the structure: 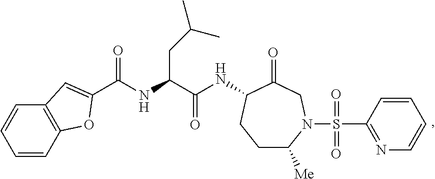 [SMILES: CC(C)C[C@H](NC(=O)c1cc2ccccc2o1)C(=O)N[C@H]1CC[C@@H](C)N(S(=O)(=O)c2ccccn2)CC1=O]
or a pharmaceutically acceptable salt thereof.

7. A method for the treatment or prophylaxis of a viral disease in a subject in need thereof, the method comprising administering to the subject a therapeutically effective amount of a first therapeutic agent that is the compound of claim 6, and administering to the subject a therapeutically effective amount of a second therapeutic agent, wherein the second therapeutic agent is not the compound of claim 6.

8. The method according to claim 7, wherein the second therapeutic agent is an agent that blocks viral entry into a cell of the subject.

9. The method according to claim 7, wherein the second therapeutic agent is an agent that acts directly on the virus, such as by modulating a viral protein.

10. The method according to claim 7, wherein the second therapeutic agent is an agent that inhibits the virus by modulating a protein, receptor or target in the subject other than a cathepsin.

11-18. (canceled)

19. The method according to claim 18, wherein the subject is a human.

20-24. (canceled)

25. The method according to claim 21, wherein the viral disease is caused by a poliovirus, a rhinovirus, a coxsackievirus (a foot-and-mouth virus (FMDV)), an influenza virus (influenza A virus, influenza B virus, influenza C virus, an avian influenza virus), a flavivirus, a hepatitis virus (hepatitis A virus, hepatitis B virus, hepatitis C virus, hepatitis D virus, hepatitis E virus), a coronavirus (SARS-CoV, SARS-CoV-2, a coronavirus responsible for Middle Eastern Respiratory Syndrome (MERS)), an Ebola virus, a Marburg virus, a Nipah virus (NiV), a Hendra virus, an arenavirus, a Rift Valley Fever virus, a yellow fever virus, a respiratory syncytial virus (RSV), a West Nile virus, a Dengue fever virus, an Aichi virus, an enterovirus, a rubella virus, a Theiler's murine encephalomyelitis virus (TMEV), an echovirus, or a human immunodeficiency virus (HIV).

26-31. (canceled)

32. The method according to claim 21, wherein the virus is an Ebola virus.

33-80. (canceled)


SEQUENCE LISTING (6 sequences)

SEQ ID NO: 1 - DNA, 20 bp, Artificial Sequence
  N1 forward primer
gaccccaaaatcagcgaaat

SEQ ID NO: 2 - DNA, 24 bp, Artificial Sequence
  N1 reverse primer
tctggttactgccagttgaatctg

SEQ ID NO: 3 - DNA, 24 bp, Artificial Sequence
  N1 Probe Sequence
accccgcattacgtttggtggacc

SEQ ID NO: 4 - DNA, 20 bp, Artificial Sequence
  N2 forward primer
ttacaaacattggccgcaaa

SEQ ID NO: 5 - DNA, 18 bp, Artificial Sequence
  N2 reverse primer
gcgcgacattccgaagaa

SEQ ID NO: 6 - DNA, 23 bp, Artificial Sequence
  N2 Probe Sequence
acaatttgcccccagcgcttcag